(19) United States
(12) Patent Application Publication
(10) Pub. No.: US 2005/0074842 A1
(43) Pub. Date: Apr. 7, 2005

(54) Human proteins having transmembrane domains and DNAs encoding these proteins

(75) Inventors: Seishi Kato, Sagamihara-shi (JP); Shingo Sekine, Ageo-shi (JP); Tomoko Kimura, Kawasaki-shi (JP)

Correspondence Address: FITZPATRICK CELLA (WYETH), 30 ROCKEFELLER PLAZA, NEW YORK, NY, 10112-3800, US

(73) Assignees: Sagami Chemical Research Center, Sagamihara-shi (JP); Protegene Inc., Meguro-ku (JP)

(21) Appl. No.: 10/626,686

(22) Filed: Jul. 25, 2003

Related U.S. Application Data
(63) Continuation of application No. 09/445,258, filed on Dec. 1, 1999, now abandoned.

(30) Foreign Application Priority Data
Jun. 3, 1997 (JP) 9-144948

(51) Int. Cl. C07K 14/705; C07H 21/04

(52) U.S. Cl. 435/69.1; 435/320.1; 435/325; 530/350; 536/23.5

(57) ABSTRACT

Proteins comprising any of the amino acid sequences of SEQ ID NOS: 1 to 18 and DNAs encoding said proteins and comprising any of the nucelotide sequences of SEQ ID NOS: 19 to 36 are provided.

### FIELD OF THE INVENTION

The present invention relates to human proteins having transmembrane domains and cDNAs encoding these proteins. The membrane proteins of this invention can be used as pharmaceuticals or as antigens for preparing antibodies against said proteins. The cDNAs of the invention can be used as probes for the gene diagnosis and gene sources for the gene therapy. The cDNAs can also be used as gene sources for large-scale production of the membrane proteins encoded by the same. The cells into which the genes encoding the membrane proteins are introduced for expression of such membrane proteins in large amounts can be used for detection of the corresponding ligands, screening of low molecular weight medicines, etc.

### BACKGROUND OF THE INVENTION

Membrane proteins play important roles as signal receptors, ion channels, transporters, etc. for the material transportation or information transmission mediated by the cell membrane. For instance, they are known to serve as receptors for various cytokines, ion channels for sodium ion, potassium ion, chloride ion, etc., transporters for saccharides and amino acids, and so on. The genes for many of them have been cloned already.

In recent years, it was clarified that the abnormalities of these membrane proteins are related to a number of hitherto cryptogenic diseases. For example, a gene for a membrane protein having 12 transmembrane domains was identified as the gene responsible for cystic fibrosis [Rommens, J. M. et al., Science 245: 1059-1065 (1989)]. It was also clarified that several membrane proteins act as the receptors when a virus infects the cells. For example, HIV-1 was revealed to infect into the cells through the mediation of a membrane protein fusin, a membrane protein on the T-cell membrane, having a CD-4 antigen and 7 transmembrane domains [Feng, Y. et al., Science 272: 872-877 (1996)]. Therefore, the discovery of newmembrane proteins is anticipated to lead to the elucidation of the causes of many diseases, and the isolation of new genes coding for the membrane proteins is desired.

Heretofore, owing to the difficulty in their purification, many of membrane proteins have been isolated by an approach from the gene side. A general method is the so-called expression cloning which comprises transfection of a cDNA library in the animal cells to express the cDNA and detection of the cells expressing the target membrane protein on the membrane by an immunological technique using an antibody or a physiological technique for the change in the membrane permeability. However, this method is applicable only to cloning of a gene for a membrane protein with a known function.

In general, membrane proteins possess hydrophobic transmembrane domains inside the proteins which are synthesized in the ribosome. Said domains remain in the phospholipid to be trapped in the membrane. Accordingly, the evidence of the cDNA for encoding the membrane protein is provided by determination of the whole base sequence of a full-length cDNA and detection of highly hydrophobic transmembrane domains in the amino acid sequence of the protein encoded by said cDNA.

As a result of the extensive study, there have successfully been obtained human proteins having transmembrane domains, particularly comprising any of the amino acid sequences of SEQ ID NOS: 1 to 18, by cloning cDNAs coding for proteins having transmembrane domains, particularly comprising any of the nucleotide sequences of SEQ ID NOS: 19 to 36, from a human full-length cDNA bank. The present invention is based on the above success.

### SUMMARY OF THE INVENTION

A main object of the present invention is to provide novel human proteins having transmembrane domains, particularly comprising any of the amino acid sequences of SEQ ID NOS: 1 to 18. Another object of this invention is to provide DNAS coding for said novel proteins, particularly comprising any of the nucleotide sequences of SEQ ID NOS: 19 to 36. A further object of the invention is to provide expression vectors capable of in vitro translating said DNAs or expressing said DNAs in eukaryotic cells. A still further object of the invention is to provide transformed eukaryotic cells capable of expressing said DNAs to produce said proteins.

In one embodiment, the present invention provides a composition comprising a protein, wherein said protein comprises an amino acid sequence selected from the group consisting of the amino acid sequences of SEQ ID NOS: 1 to 18 and their fragments.

In another embodiment, the present invention provides a composition comprising an isolated polynucleotide selected from the group consisting of the nucleotide sequences of SEQ ID NOS: 19 to 36.

In a further embodiment, the present invention provides a composition comprising an isolated polynucleotide selected from the group consisting of the nucleotide sequences of SEQ ID NOS: 37 to 54.

### BRIEF DESCRIPTION OF DRAWINGS

FIG. 1: A figure depicting the structure of the secretory signal sequence detection vector pSSD3.

FIG. 2: A figure depicting the hydrophobicity/hydrophilicity profile of the protein encoded by clone HP01263.

FIG. 3: A figure depicting the hydrophobicity/hydrophilicity profile of the protein encoded by clone HP01299.

FIG. 4: A figure depicting the hydrophobicity/hydrophilicity profile of the protein encoded by clone HP01347.

FIG. 5: A figure depicting the hydrophobicity/hydrophilicity profile of the protein encoded by clone HP01440.

FIG. 6: A figure depicting the hydrophobicity/hydrophilicity profile of the protein encoded by clone HP01526.

FIG. 7: A figure depicting the hydrophobicity/hydrophilicity profile of the protein encoded by clone HP10230.

FIG. 8: A figure depicting the hydrophobicity/hydrophilicity profile of the protein encoded by clone HP10389.

FIG. 9: A figure depicting the hydrophobicity/hydrophilicity profile of the protein encoded by clone HP10408.

FIG. 10: A figure depicting the hydrophobicity/hydrophilicity profile of the protein encoded by clone HP10412.

FIG. 11: A figure depicting the hydrophobicity/hydrophilicity profile of the protein encoded by clone HP10413.

FIG. 12: A figure depicting the hydrophobicity/hydrophilicity profile of the protein encoded by clone HP10415.

FIG. 13: A figure depicting the hydrophobicity/hydrophilicity profile of the protein encoded by clone HP10419.

FIG. 14: A figure depicting the hydrophobicity/hydrophilicity profile of the protein encoded by clone HP10424.

FIG. 15: A figure depicting the hydrophobicity/hydrophilicity profile of the protein encoded by clone HP10428.

FIG. 16: A figure depicting the hydrophobicity/hydrophilicity profile of the protein encoded by clone HP10429.

FIG. 17: A figure depicting the hydrophobicity/hydrophilicity profile of the protein encoded by clone HP10432.

FIG. 18: A figure depicting the hydrophobicity/hydrophilicity profile of the protein encoded by clone HP10433.

FIG. 19: A figure depicting the hydrophobicity/hydrophilicity profile of the protein encoded by clone HP10480.

### BEST MODE FOR CARRING OUT INVENTION

The proteins of the present invention can be obtained, for example, by isolation from human organs, cell lines, etc., by chemical synthesis on the basis of the amino acid sequences as herein disclosed, or by recombinant DNA technology using the DNA encoding the transmembrane domains of the invention. Among them, adoption of the recombinant DNA technology is preferred. Specifically, each of the proteins may be prepared by in vitro transcription of a vector comprising the cDNA of the invention to make RNA and in vitro translation using this RNA as a template to accomplish in vitro expression. Also, each of the proteins may be prepared in a large amount by the use of Escherichia coli, Bacillus subtilis, yeasts, animal cells, etc. comprising a suitable expression vector having the DNA encoding such protein.

In the case of producing the protein of the invention by the use of a microorganism such as Escherichia coli, the translation region of the cDNA of the invention is constructed in an expression vector having an origin, a promoter, a ribosome-binding site, a cDNA-cloning site, a terminator, etc. that can be replicated in the microorganism and, after transformation of the host cells with said expression vector, the resultant transformant is incubated, whereby the protein encoded by said cDNA can be produced in a large amount in the microorganism. In that case, a protein fragment containing an optional region can be obtained by performing the expression with inserting an initiation codon and a termination codon before and after the optional translation region. Alternatively, a fusion protein with another protein can be expressed. Only a protein portion encoding said cDNA can be obtained by cleavage of said fusion protein with an appropriate protease.

For production of the protein of the invention by expression of DNA coding for such protein in eukaryotic cells, the translation region of said cDNA may be recombined into an expression vector for eukaryotic cells having a promoter, a splicing domain, a poly(A) addition site, etc., followed by introduction into eukaryotic cells so that the protein of the invention is produced as a membrane protein on the cell membrane surface. Examples of the expression vector are pKA1, pED6_dpc2, pCDM8, pSVK3, PMSG, pSVL, PBK-CMV, pBK-RSV, EBV vector, pRS, pYES2, etc. As the eukaryotic cells, there are exemplified mammalian animal culture cells (e.g. simian kidney cells COS7, chinese hamster ovary cells CHO), budding yeasts, Schizosaccharomyces pombe, silkworm cells, Xenopus laevis egg cells, etc., but any other eukaryotic cells may also be used insofar as the protein of the invention can be expressed on the membrane surface. In order to introduce the expression vector into eukaryotic cells, there may be adopted any conventional procedure such as electroporation, calcium phosphate method, liposome method or DEAE dextran method.

The proteins of the present invention include peptide fragments (5 or more amino acid residues) containing any partial amino acid sequence of the amino acid sequences of SEQ ID NOS: 1 to 18. These fragments can be used as antigens for preparation of the antibodies. Also, the proteins of the invention that have signal sequences appear in the form of maturation proteins on the cell surface, after the signal sequences are removed. Therefore, these maturation proteins shall come within the scope of the present invention. The N-terminal amino acid sequences of the maturation proteins can be easily identified by using the method for the cleavage-site determination in a signal sequence [Japan Patent Kokai No. 187100/96]. Further, many membrane proteins are subjected to the processing on the cell surface to be converted to the secretor forms. These secretor proteins or peptides shall come within the scope of the present invention. When glycosylation sites are present in the amino acid sequences, expression in appropriate animal cells affords glycosylated proteins. Therefore, these glycosylated proteins or peptides also shall come within the scope of the invention.

The DNAs of the invention include all DNAs encoding the above-mentioned proteins. Said DNAs can be obtained using the method by chemical synthesis, the method by cDNA cloning, and so on.

Each of the cDNAs of the invention can be cloned from, for example, the cDNA libraries of the human cell origin. The cDNA is synthesized using as a template a poly(A)⁺ RNA extracted from human cells. The human cells may be cells delivered from the human body, for example, by the operation or may be the culture cells. The cDNA can be synthesized by using any method selected from the Okayama-Berg method [Okayama, H. and Berg, P., Mol. Cell. Biol. 2: 161-170 (1982)], the Gubler-Hoffman method [Gubler, U. and Hoffman, J. Gene 25: 263-269 (1983)], and so on, but it is preferred to use the capping method [Kato, S. et al., Gene 150: 243-250 (1994)] as illustrated in Examples in order to obtain a full-length clone in an effective manner.

The primary selection of a cDNA encoding a human protein having transmembrane domains is performed by the sequencing of a partial base sequence of the cDNA clone selected at random from the cDNA libraries, sequencing of the amino acid sequence encoded by the base sequence, and recognition of the presence or absence of hydrophobic site(s) in the resulting N-terminal amino acid sequence region. Next, the secondary selection is carried out by determination of the whole base sequence by the sequencing and the protein expression by the in vitro translation. The ascertainment of the cDNA of the present invention for encoding the protein having the secretory signal sequence is performed by using the signal sequence detection method [Yokoyama-Kobayashi, M. et al., Gene 163: 193-196 (1995)]. In other words, the ascertainment for the coding portion of the inserted cDNA fragment to function as a signal sequence is provided by fusing a cDNA fragment encoding the N-terminus of the target protein with a cDNA encoding the protease domain of urokinase and then expressing the resulting cDNA in COS7 cells to detect the urokinase activity in the cell culture medium. On the other hand, the N-terminal region is judged to remain in the membrane in the case where the urokinase activity is not detected in the cell culture medium.

The cDNAs of the invention are characterized by containing any of the nucleotide sequences of SEQ ID NOS: 19 to 36 or any of the nucleotide sequences of SEQ ID NOS: 37 to 54. Table 1 summarizes the clone number (HP number), the cells affording the cDNA, the total nucleotide number of the cDNA, and the number of the amino acid residues of the encoded protein, for each of the cDNAs.

TABLE 1

| | | | | Number of |
|---|---|---|---|---|
| Sequence | HP | | Number of | Amino Acid |
| Number | Number | Cells | Nucleotides | Residues |
| 1, 19, 37 | HP01263 | Liver | 1502 | 382 |
| 2, 20, 38 | HP01299 | Liver | 1349 | 317 |
| 3, 21, 39 | HP01347 | Liver | 1643 | 296 |
| 4, 22, 40 | HP01440 | Stomach cancer | 729 | 197 |
| 5, 23, 41 | HP01526 | Stomach cancer | 1322 | 221 |
| 6, 24, 42 | HP10230 | Stomach cancer | 3045 | 251 |
| 7, 25, 43 | HP10389 | KB | 653 | 106 |
| 8, 26, 44 | HP10408 | Stomach cancer | 439 | 78 |
| 9, 27, 45 | HP10412 | Stomach cancer | 1131 | 314 |
| 10, 28, 46 | HP10413 | Stomach cancer | 1875 | 195 |
| 11, 29, 47 | HP10415 | Stomach cancer | 1563 | 462 |
| 12, 30, 48 | HP10419 | Stomach cancer | 2030 | 247 |
| 13, 31, 49 | HP10424 | Stomach cancer | 493 | 113 |
| 14, 32, 50 | HP10428 | KB | 2044 | 365 |
| 15, 33, 51 | HP10429 | Stomach cancer | 1043 | 226 |
| 16, 34, 52 | HP10432 | Liver | 972 | 129 |
| 17, 35, 53 | HP10433 | Liver | 695 | 163 |
| 18, 36, 54 | HP10480 | Stomach cancer | 1914 | 193 |

Hereupon, the same clone as any of the cDNAs of the invention can be easily obtained by screening of the cDNA libraries constructed from the cell line or the human tissues employed in the invention, by the use of an oligonucleotide probe synthesized on the basis of the corresponding cDNA nucleotide sequence of SEQ ID NOS: 37 to 54.

In general, the polymorphism due to the individual difference is frequently observed in human genes. Therefore, any cDNA that is subjected to insertion or deletion of one or plural nucleotides and/or substitution with other nucleotides in SEQ ID NOS: 37 to 54 shall come within the scope of the invention.

In a similar manner, any protein that is produced by these modifications comprising insertion or deletion of one or plural nucleotides and/or substitution with other nucleotides shall come within the scope of the present invention, as far as said protein possesses the activity of the corresponding protein having the amino acid sequence of SEQ ID NOS: 1 to 18.

The cDNAs of the invention include cDNA fragments (more than 10 bp) containing any partial nucleotide sequence of the nucleotide sequence of SEQ ID NOS: 19 to 36 or of the nucleotide sequence of SEQ ID NOS: 37 to 54. Also, DNA fragments consisting of a sense chain and an anti-sense chain shall come within this scope. These DNA fragments can be used as the probes for the gene diagnosis.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614, 396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information.

Proteins and protein fragments of the present invention include proteins with amino acid sequence lengths that are at least 25%(more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologs of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide, as determined by those of skill in the art. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotide which also encode proteins which are identical, homologous, or related to that encoded by the polynucleotides.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2

| | | Hybrid | | Wash |
|---|---|---|---|---|
| Stringency | Polynucleotide | Length | Hybridization Temperature | Temperature |
| Condition | Hybrid | (bp)^{‡} | and Buffer^{†} | and Buffer^{†} |
| A | DNA:DNA | ≧50 | 65° C.; 1 × SSC -or- | 65° C.; 0.3 × SSC |
| | | | 42° C.; 1 × SSC, 50% formamide | |
| B | DNA:DNA | <50 | T_{B}*; 1 × SSC | T_{B}*; 1 × SSC |
| C | DNA:RNA | ≧50 | 67° C.; 1 × SSC -or- | 67° C.; 0.3 × SSC |
| | | | 45° C.; 1 × SSC, 50% formamide | |
| D | DNA:RNA | <50 | T_{D}*; 1 × SSC | T_{D}*; 1 × SSC |
| E | RNA:RNA | ≧50 | 70° C.; 1 × SSC -or- | 70° C.; 0.3 × SSC |
| | | | 50° C.; 1 × SSC, 50% formamide | |
| F | RNA:RNA | <50 | T_{F}*; 1 × SSC | T_{F}*; 1 × SSC |
| G | DNA:DNA | ≧50 | 65° C.; 4 × SSC -or- | 65° C.; 1 × SSC |
| | | | 42° C.; 4 × SSC, 50% formamide | |
| H | DNA:DNA | <50 | T_{H}*; 4 × SSC | T_{H}*; 4 × SSC |
| I | DNA:RNA | ≧50 | 67° C.; 4 × SSC -or- | 67° C.; 1 × SSC |
| | | | 45° C.; 4 × SSC, 50% formamide | |
| J | DNA:RNA | <50 | T_{J}*; 4 × SSC | T_{J}*; 4 × SSC |
| K | RNA:RNA | ≧50 | 70° C.; 4 × SSC -or- | 67° C.; 1 × SSC |
| | | | 50° C.; 4 × SSC, 50% formamide | |
| L | RNA:RNA | <50 | T_{L}*; 2 × SSC | T_{L}*; 2 × SSC |
| M | DNA:DNA | ≧50 | 50° C.; 4 × SSC -or- | 50° C.; 2 × SSC |
| | | | 40° C.; 6 × SSC, 50% formamide | |
| N | DNA:DNA | <50 | T_{N}*; 6 × SSC | T_{N}*; 6 × SSC |
| O | DNA:RNA | ≧50 | 55° C.; 4 × SSC -or- | 55° C.; 2 × SSC |
| | | | 42° C.; 6 × SSC, 50% formamide | |
| P | DNA:RNA | <50 | T_{P}*; 6 × SSC | T_{P}*; 6 × SSC |
| Q | RNA:RNA | ≧50 | 60° C.; 4 × SSC -or- | 60° C.; 2 × SSC |
| | | | 45° C.; 6 × SSC, 50% formamide | |
| R | RNA:RNA | <50 | T_{R}*; 4 × SSC | T_{R}*; 4 × SSC |
| ^{‡}The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When | | | | |
| # polynucleotides of known sequence are hybridized, the hybrid length can be determined by aligning the sequences of the polynucleotides and identifying the region or regions of optimal sequence complementarity. | | | | |
| ^{†}SSPE (1 × SSPE is 0.15M NaCl, 10 mM NaH₂PO₄, and 1.25 mM EDTA, pH7.4) can be substituted for SSC (1 × SSC is 0.15M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, | | | | |
| # Tₘ(° C.) = 2(# of A + T bases) + 4(# of G + C bases). For hybrids between 18 and 49 base pairs in length. Tₘ(° C.) = 81.5 + 16.6(log₁₀[Na⁺]) + 0.41 (% G + C) − (600/N), where N is the number of bases in the hybrid, and [Na⁺] is the concentration of sodium ions in the hybridization buffer ([Na⁺] for 1 × SSC = 0.165M). | | | | |

Additional examples of stringency conditions for polynucleotide hybridization are provided in Sambrook, J., E. F. Fritsch, and T. Maniati's, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25%(more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

### EXAMPLE

The present invention is embodied in more detail by the following examples, but this embodiment is not intended to restrict the present invention. The basic operations and the enzyme reactions with regard to the DNA recombination are carried out according to the literature [“Molecular Cloning. A Laboratory Manual”, Cold Spring Harbor Laboratory, 1989]. Unless otherwise stated, restrictive enzymes and a variety of modification enzymes to be used were those available from Takara Shuzo Co., Ltd. The manufacturer's instructions were used for the buffer compositions as well as for the reaction conditions, in each of the enzyme reactions. The cDNA synthesis was carried out according to the literature [Kato, S. et al., Gene 150: 243-250 (1994)].

(1) Preparation of Poly(A)⁺ RNA

The epidermoid carcinoma cell line KB (ATCC CRL 17), tissues of stomach cancer delivered by the operation, and liver were used for human cells to extract mRNAs. The cell line was cultured by a conventional procedure.

After about 1 g of human tissues was homogenized in 20 ml of a 5.5 M guanidinium thiocyanate solution, total mRNAs were prepared in accordance with the literature [Okayama, H. et al., “Methods in Enzymology” Vol. 164, Academic Press, 1987]. These mRNAs were subjected to chromatography using an oligo(dT)-cellulose column washed with 20 mM Tris-hydrochloric acid buffer solution (pH 7.6), 0.5 M NaCl, and 1 mM EDTA to obtain a poly(A)+ RNA in accordance with the above-mentioned literature.

(2) Construction of cDNA Library

To a solution of 10 μg of the above-mentioned poly(A)⁺ RNA in 100 mM Tris-hydrochloric acid buffer solution (pH 8) was added one unit of an RNase-free, bacterium-origin alkaline phosphatase and the resulting solution was allowed to react at 37° C. for one hour. After the reaction solution underwent the phenol extraction followed by the ethanol precipitation, the obtained pellets were dissolved in a mixed solution of 50 mM sodium acetate (pH 6), 1 mM EDTA, 0.1% 2-mercaptoethanol, and 0.01% Triton X-100. Thereto was added one unit of a tobacco-origin pyrophosphatase (Epicenter Technologies) and the resulting solution at a total volume of 100 μl was allowed to react at 37° C. for one hour. After the reaction solution underwent the phenol extraction followed by the ethanol precipitation, the thus-obtained pellets were dissolved in water to obtain a decapped poly(A)⁺ RNA solution.

To a solution of the decapped poly(A)⁺ RNA and 3 nmol of a DNA-RNA chimeric oligonucleotide (5′-dG-dG-dG-dG-dA-dA-dT-dT-dC-dG-dA-G-G-A-3′) in a mixed aqueous solution of 50 mM Tris-hydrochloric acid buffer solution (pH 7.5), 0.5 mM ATP, 5 mM MgCl₂, 10 mM 2-mercaptoethanol, and 25% polyethylene glycol were added 50 units of T4 RNA ligase and the resulting solution at a total volume of 30 μl was allowed to react at 20° C. for 12 hours. After the reaction solution underwent the phenol extraction followed by the ethanol precipitation, the thus-obtained pellets were dissolved in water to obtain a chimeric oligo-capped poly(A)⁺ RNA.

After the vector pKA1 developed by the present inventors (Japanese Patent Kokai Publication No. 1992-117292) was digested with KpnI, an about 60-dT tail was inserted by a terminal transferase. This product was digested with EcoRV to remove the dT tail at one side and the resulting molecule was used as a vectorial primer.

After 6 μg of the previously-prepared chimeric oligo-capped poly(A)⁺ RNA was annealed with 1.2 μg of the vectorial primer, the product was dissolved in a mixed solution of 50 mM Tris-hydrochloric acid buffer solution (pH 8.3), 75 mM KCl, 3 mM MgCl₂₁ 10 mM dithiothreitol, and 1.25 mM dNTP (DATP+dCTP+dGTP+dTTP), mixed with 200 units of a reverse transferase (GIBCO-BRL), and the resulting solution at a total volume of 20 μl was allowed to react at 42° C. for one hour. After the reaction solution underwent the phenol extraction followed by the ethanol precipitation, the thus-obtained pellets were dissolved in a mixed solution of 50 mM Tris-hydrochloric acid buffer solution (pH 7.5), 100 mM NaCl, 10 MM MgCl₂, and 1 mM dithiothreitol. Thereto were added 100 units of EcoRI and the resulting solution at a total volume of 20 μl was allowed to react at 37° C. for one hour. After the reaction solution underwent the phenol extraction followed by the ethanol precipitation, the obtained pellets were dissolved in a mixed solution of 20 mM Tris-hydrochloric acid buffer solution (pH 7.5), 100 mM KCl, 4 MM MgCl₂, 10 mM (NH₄)₂SO₄, and 50 μg/ml bovine serum albumin. Thereto were added 60 units of Escherichia coli DNA ligase and the resulting solution was allowed to react at 16° C. for 16 hours. To the reaction solution were added 2 μl of 2 mM dNTP, 4 units of Escherichia coli DNA polymerase I, and 0.1 unit of Escherichia coli DNase H and the resulting solution was allowed to react at 12° C. for one hour and then at 22° C. for one hour.

Next, the cDNA-synthesis reaction solution was used to transform Escherichia coli DH12S (GIBCO-BRL). The transformation was carried out by the electroporation method. A portion of the transformant was inoculated on a 2xYT agar culture medium containing 100 μg/ml ampicillin, which was incubated at 37° C. overnight. A colony grown on the culture medium was randomly picked up and inoculated on 2 ml of the 2xYT culture medium containing 100 μg/ml ampicillin, which was incubated at 37° C. overnight. The culture medium was centrifuged to separate the cells, from which a plasmid DNA was prepared by the alkaline lysis method. After the plasmid DNA was double-digested with EcoRI and NotI, the product was subjected to 0.8% agarose gel electrophoresis to determine the size of the cDNA insert. In addition, by the use of the obtained plasmid as a template, the sequence reaction using M13 universal primer labeled with a fluorescent dye and Taq polymerase (a kit of Applied Biosystems Inc.) was carried out and the product was analyzed by a fluorescent DNA-sequencer (Applied Biosystems Inc.) to determine the base sequence of the cDNA 5′-terminal of about 400 bp. The sequence data were filed as a homo-protein cDNA bank data base.

(3) Selection of cDNAs Encoding Proteins Having Transmembrane Domains

The base sequence registered in the homo-protein cDNA bank data base was converted to three frames of amino acid sequences and the presence or absence of an open reading frame (ORF) beginning from the initiation codon. Then, the selection was made for the presence of a signal sequence that is characteristic to a secretory protein at the N-terminal of the portion encoded by ORF. These clones were sequenced from the both 5′ and 3′ directions by using the deletion method to determine the sequence of the whole base sequence. The hydrophobicity/hydrophilicity profiles were obtained for proteins encoded by ORF by the Kyte-Doolittle method [Kyte, J. & Doolittle, R. F., J. Mol. Bio. 157: 105-132 (1982)] to examine the presence or absence of a hydrophobic region. In the case in which there is a hydrophobic region of putative transmembrane domain(s) in the amino acid sequence of an encoded protein, this protein was considered as a membrane protein.

(4) Construction of Secretory Signal Detection Vector pSSD3

One microgram of pSSDl carrying the SV40 promoter and a cDNA encoding the protease domain of urokinase [Yokoyama-Kobayashi, M. et al., Gene 163: 193-196 (1995)] was digested with 5 units of BglII and 5 units of EcoRV. Then, after dephosphorylation at the 5′ terminal by the CIP treatment, a DNA fragment of about 4.2 kbp was purified by cutting off from the gel of agarose gel electrophoresis.

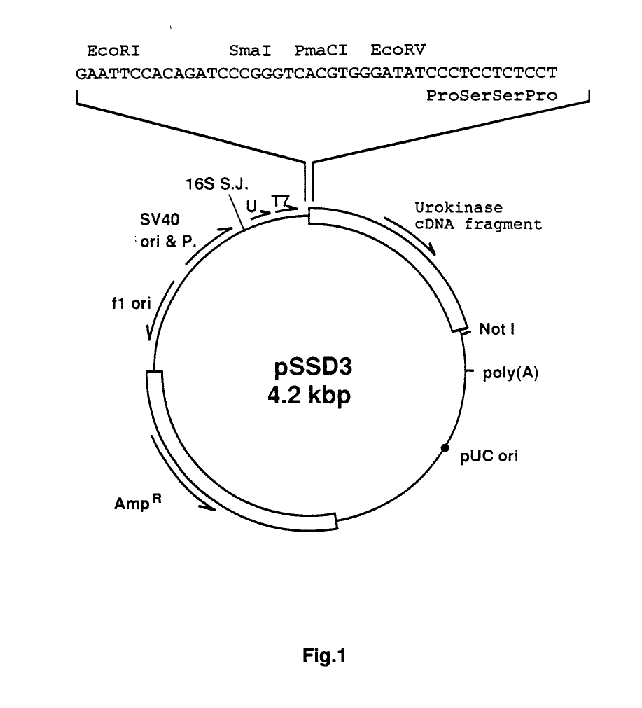

Two oligo DNA linkers, L1 (5′-GATCCCGGGTCACGTGGGAT-3′) and L2 (5′-ATCCCACGTGACCCGG-3′), were synthesized and phosphorylated by T4 polynucleotide kinase. After annealing of the both linkers, followed by ligation with the previously-prepared pSSDl fragment by T4 DNA ligase, Escherichia coli JM109 was transformed. A plasmid pSSD3 was prepared from the transformant and the objective recombinant was confirmed by the determination of the base sequence of the linker-inserted fragment. FIG. 1 illustrates the structure of the thus-obtained plasmid. The present plasmid vector carries three types of blunt-end formation restriction enzyme sites, SmaI, PmaCI, and EcoRV. Since these cleavage sites are positioned in succession at an interval of 7 bp, selection of an appropriate site in combination of three types of frames for the inserting cDNA allows to construct a vector expressing a fusion protein.

(5) Functional Verification of Secretory Signal Sequence

Whether the N-terminal hydrophobic region in the secretory protein clone candidate obtained in the above-mentioned steps functions as the secretory signal sequence was verified by the method described in the literature [Yokoyama-Kobayashi, M. et al., Gene 163: 193-196 (1995)]. First, the plasmid containing the target cDNA was cleaved at an appropriate restriction enzyme site that existed at the downstream of the portion expected for encoding the secretory signal sequence. In the case in which this restriction enzyme site was a protruding terminus, the site was blunt-ended by the Klenow treatment or treatment with the mung-bean nuclease. Digestion with HindIII was further carried out and a DNA fragment containing the SV40 promoter and a cDNA encoding the secretory sequence at the downstream of the promoter was separated by agarose gel electrophoresis. This fragment was inserted between the pSSD3 HindIII site and a restriction enzyme site selected so as to match with the urokinase-coding frame, thereby constructing a vector expressing a fusion protein of the secretory signal portion of the target cDNA and the urokinase protease domain.

After Escherichia coli (host: JM109) bearing the fusion-protein expression vector was incubated at 37° C. for 2 hours in 2 ml of the 2xYT culture medium containing 100 μg/ml ampicillin, the helper phage M13KO7 (50 μl) was added and the incubation was continued at 37° C. overnight. A supernatant separated by centrifugation underwent precipitation with polyethylene glycol to obtain single-stranded phage particles. These particles were suspended in 100 μl of 1 mM Tris-0.1 mM EDTA, pH 8 (TE). Also, there was used as a control a suspension of single-stranded particles prepared in the same manner from the vector pLAl-UPA containing pSSD3 and a full-length cDNA of urokinase [Yokoyama-Kobayashi, M. et al., Gene 163: 193-196 (1995)].

The simian-kidney-origin culture cells, COS7, were incubated at 37° C. in the presence of 5% CO₂ in the Dulbecco's modified Eagle's culture medium (DMEM) containing 10% bovine fetus albumin. Into a 6-well plate (Nunc Inc., 3 cm in the well diameter) were inoculated 1×10⁵ COS7 cells and incubation was carried out at 37° C. for 22 hours in the presence of 5% CO₂. After the culture medium was removed, the cell surface was washed with a phosphate buffer solution and then washed again with DMEM containing 50 mM Tris-hydrochloric acid (pH 7.5) (TDMEM). To the cells were added 1 μl of the single-stranded phage suspension, 0.6 ml of the DMEM culture medium, and 3 μl of TRANSFECTAM™ (IBF Inc.) and the resulting mixture was incubated at 37° C. for 3 hours in the presence of 5% CO₂. After the sample solution was removed, the cell surface was washed with TDMEM, 2 ml per well of DMEM containing 10% bovine fetus albumin was added, and the incubation was carried out at 37° C. for 2 days in the presence of 5% CO₂.

To 10 ml of 50 mM phosphate buffer solution (pH 7.4) containing 2% bovine fibrinogen (Miles Inc.), 0.5% agarose, and 1 mM potassium chloride were added 10 units of human thrombin (Mochida Pharmaceutical Co., Ltd.) and the resulting mixture was solidified in a plate of 9 cm in diameter to prepare a fibrin plate. Ten microliters of the culture supernatant of the transfected COS7 cells were spotted on the fibrin plate, which was incubated at 37° C. for 15 hours. The diameter of the thus-obtained clear circle was taken as an index for the urokinase activity. In the case in which a cDNA fragment codes for the amino acid sequence that functions as a secretory signal sequence, a fusion protein is secreted to form a clear circle by its urokinase activity. Therefore, in the case in which a clear circle is not formed, the fusion protein remains as trapped in the membrane and the cDNA fragment is considered to code for a transmembrane domain.

(6) Protein Synthesis by In Vitro Translation

The plasmid vector carrying the cDNA of the present invention was utilized for the transcription/translation by the T_{N}T rabbit reticulocyte lysate kit (Promega Biotec). In this case, [³⁵S]methionine was added and the expression product was labeled with the radioisotope. All reactions were carried out by following the protocols attached to the kit. Two micrograms of the plasmid was allowed to react at 30° C. for 90 minutes in total 25 ml of a reaction solution containing 12.5 μl of the T_{N}T rabbit reticulocyte lysate, 0.5 μl of the buffer solution (attached to the kit), 2 μl of an amino acid mixture (methionine-free), 2 μl (0.37 MBq/μl) of [³⁵S]methionine (Amersham Corporation), 0.5 μl of T7 RNA polymerase, and 20 U of RNasin. To 3 μl of the reaction solution was added 2 μl of an SDS sampling buffer (125 mM Tris-hydrochloric acid suffer solution, pH 6.8, 120 mM 2-mercaptoethanol, 2% SDS solution, 0.025% bromophenol blue, and 20% glycerol) and the resulting solution was heated at 95° C. for 3 minutes and then subjected to SDS-polyacrylamide gel electrophoresis. The molecular weight of the translation product was determined by carrying out the autoradiography.

(7) Expression in COS7

Escherichia coli bearing a vector expressing the protein of the invention was infected with helper phage M13KO7, and single-stranded phage particles were obtained according to the method as stated above. Using the thus obtained phages, each expression vecotr was introduced into simian-kidney-origin culture cells COS7 in the manner as stated above. After incubation at 37° C. for 2 days in the presence of 5% CO₂, further incubation was carried out in a medium containing [³⁵S]cysteine or [³⁵S]methionine for 1 hour. The cells were collected, dissolved and then subjected to SDS-PAGE whereby a band corresponding to the expression product of each protein which is not present in COS7 cells was revealed. In Table 3, the molecular weight of each expression product is shown.

TABLE 3

| | Supernatant of culture | Membrane fraction |
|---|---|---|
| HP Number | (kDa) | (kDa) |
| HP01263 | 50 | — |
| HP01299 | — | 30 |
| HP01526 | — | 22 |
| HP10230 | — | 24 |
| HP10408 | — | 7 |
| HP10415 | — | 45 |
| HP10424 | — | 14 |
| HP10429 | — | 27 |
| HP10432 | — | 17 |
| HP10480 | — | 22 |

(8) Clone Examples

<HP01263> (Sequence Number 1, 19, 37)

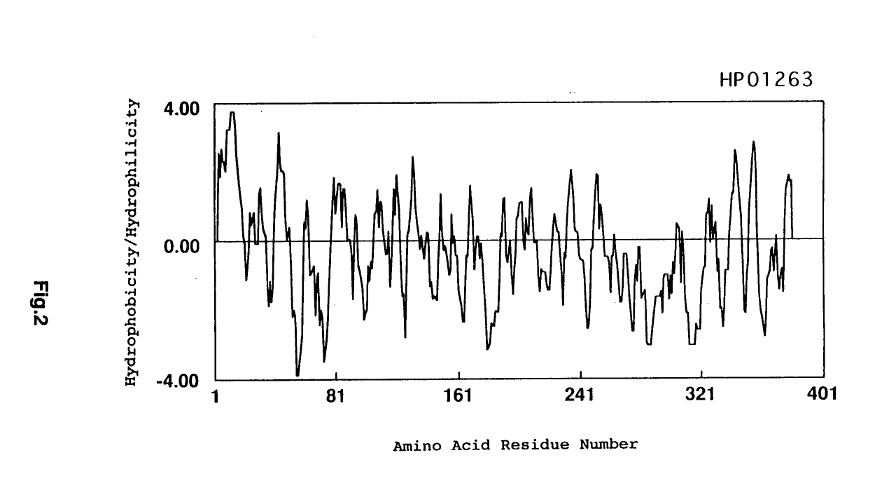

Determination of the whole base sequence for the cDNA insert of clone HP01263 obtained from the human liver cDNA libraries revealed the structure consisting of a 5′-non-translation region of 36 bp, an ORF of 1149 bp, and a 3′-non-translation region of 316 bp. The ORF codes for a protein consisting of 382 amino acid residues with one transmembrane domain at the N-terminal. FIG. 2 depicts the hydrophobicity/hydrophilicity profile of the present protein obtained by the Kyte-Doolittle method. The in vitro translation resulted in formation of a translation product of 42 kDa, which is almost consistent with the molecular weight of 42,054 as predicted from the ORF. On expression in COS cells, an expression product of about 50 kDa was observed in the culture supernatant. Therefore, said protein can be understood to be a secreted protein. Application of the rule (−3, −1) as a method for anticipation of a cutting site in a secretion signal sequence suggested that the mature protein would start from methionine at 19 position.

The search of the protein data base using the amino acid sequence of the present protein revealed that the protein was analogous to the human α-2-HS-glycoprotein (SWISS-PROT Accession No. P02765). Table 4 indicates the comparison of the amino acid sequences between the human protein of the present invention (HP) and the human α-2-HS-glycoprotein (GP). - represents a gap, * represents an amino acid residue identical to that in the protein of the present invention, and . represents an amino acid residue analogous to that in the protein of the present invention. The both proteins possessed a homology of 25.5%. The cysteine position is reserved and this region is analogous to that in cystatins (thiol proteinase inhibitors). There are observed other analogy with histidine-rich glycoprotein (P04196, 30.9%/194 amino acid residues), kininogen (P01045, 24.1%/261 amino acid residues), tyrosine kinase inhibitor (A32827, 24.4%/291 amino acid residues), and so on.

TABLE 4

| | |
|---|---|
| HP | MGLLLPLALCILVLCCGAMSPPQLALNPSALLSR--GCNDSDVLAVAGFALRDINKDRKD |
| | .*.** ... . * . ..*. * .*.*... ..* *. **.. |
| GP | MKSLVLLLCLAQLWGCHSAPHGPGLIYRQPNCDDPETEEAALVAIDYINQNLPW |
| HP | GYVLRLNRVNDAQEYRRGGLGSLFYLTLDVLETDCHVLRKKAWQDCGMRIFFE-SVYGQC |
| | ** **...... ... *.** ...*.***.**** .. . *..* . * .* *.* |
| GP | GYKHTLNQIDEVKVWPQQPSGELFEIEIDTLETTCHVLDPTPVARCSVRQLKEHAVEGDC |
| HP | K-AIFYMNNPSRVLYLAAYNCTLRPVSKKKIYMTCPDCPSSIPTDSSNHQVLEAATESLA |
| | . ... ... .*.* .*. .* * ... ..*.*** * . . .*..**...** |
| GP | DFQLLKLDGKFSVVY---AKCDSSPDSAEDVRKVCQDCPLLAPLN--DTRVVHAAKAALA |
| HP | KYNNENTSKQYSLFKVTRASSQWVVGPSYFVEYLIKESPC---TKSQASSCSLQSSDSVP |
| | .*..*..... * ...** . ** .**. .... * . ..*..*.* ... |
| GP | AFNAQNNGSNFQLEEISRAQLV-PLPPSTYVEFTVSGTDCVAKEATEAAKCNLLAEKQY- |
| HP | VGLCKGSLTRTHWEKFVSVTCDFFESQAPATGSENSAVNQK-PTNLPKVEESQQKNTPPT |
| | *.**..*. . . . *.***. *..*. .. .. ...*. ** . . .... .. |
| GP | -GFCKATLSEKLGGAEVAVTCTVFQTQPVTSQPQPEGANEAVPTPVVDPDAPPSPPLGAP |
| HP | DSPSKAGPRGSVQYLPDLDDKNSQEKGPQEAFPVHLDLTTNPQGETLDISFLFLEPMEEK |
| | . *. ..*..* *. |
| GP | GLPPAGSPPDSRVLLAAPPGHQLHRAHYDLRHTFMGVVSLGSPSGEVSHPRKTRTVVQPS |
| HP | LVVLPFPKEKARTAECPGPAQNASPLVLPP |
| GP | VGAAAGPVVPPCPGRIRHFKV |

Furthermore, the search of GenBank using the base sequence of the present cDNA revealed that there existed some ESTs possessing the homology of 90% or more (for example, Accession No. H57204), but it can not be assessed whether these ESTs with partial sequences code for the same protein as the protein of the present invention. Hereupon, most of ESTs matching with the present cDNA are available from liver cDNA libraries, whereby the present clone is considered to be expressed specifically in the liver.

The present protein, because of being a type-II membrane protein, is considered to exert its function as a receptor on the membrane surface with the C-terminal side exposed outside the cells or after undergoing a processing followed by being excreted in the serum. The present protein, because of bearing a cystatin-like domain, is considered to possess a proteinase-inhibitor activity as well as many physiological activities in the same manner as for other members of this family. In addition, the present protein, because of being expressed specifically in liver cells, is considered to play a significant role for maintaining the liver function.

<HP01299> (Sequence Number 2, 20, 38)

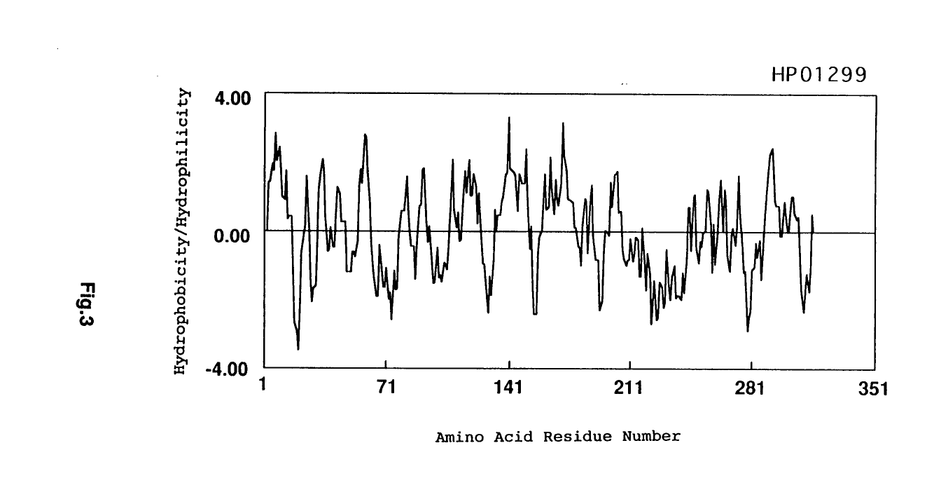

Determination of the whole base sequence for the cDNA insert of clone HP01299 obtained from the human liver cDNA libraries revealed the structure consisting of a 5′-non-translation region of 110 bp, an ORF of 954 bp, and a 3′-non-translation region of 285 bp. The ORF codes for a protein consisting of 317 amino acid residues with two or more transmembrane domains. FIG. 3 depicts the hydrophobicity/hydrophilicity profile of the present protein obtained by the Kyte-Doolittle method. The in vitro translation resulted in the formation of a translation product of 32 kDa that was almost consistent with the molecular weight of 35,965 predicted from the ORF.

The search of the protein data base using the amino acid sequence of the present protein revealed that the protein was analogous to the rat retinol dehydrogenase (NBRF Accession No. A55884). Table 5 indicates the comparison of the amino acid sequences between the human protein of the present invention (HP) and the rat retinol dehydrogenase (RN). - represents a gap, * represents an amino acid residue identical to that in the protein of the present invention, and . represents an amino acid residue analogous to that in the protein of the present invention. The both proteins possessed a homology of 65.3% among the entire regions.

TABLE 5

| | |
|---|---|
| HP | MWLYLAAFVGLYYLLHWYRERQVVSHLQDKYVFITGCDSCFGNLLARQLDARGLRVLAAC |
| | ***** *.***. **. .***.**************************** **.****** |
| RN | MWLYLLALVGLWNLLRLFRERKVVSHLQDKYVFITGCDSGFGNLLARQLDRRGMRVLAAC |
| HP | LTEKGAEQLRGQTSDRLETVTLDVTKMESIAAATQWVKEHVGDRGLWGLVNNAGILTPIT |
| | **********..********.***** ***.********.**.************ .*.. |
| RN | LTEKGAEQLRSKTSDRLETVILDVTKTESIVAATQWVKERVGNRGLWGLVNNAGISVPVQ |
| HP | LCEWLNTEDSMNMLKVNLIGVIQVTLSMLPLVRRARGRIVNVSSILGRVAFFVGGYCVSK |
| | **....* ..*.***.***.***.******.****.**..*..**... ****.** |
| RN | PNEWMRKKDFASVLDVNLLGVIEVTLNMLPLVRKARGRVVNIASTMGRMSLVGGGYCISK |
| HP | YGVEAFSDILRREIQHFGVKISIVEPGYFRTGMTNMTQSLERMKQSWKEAPKHIKETYGQ |
| | ******** ****. .****..*.*** *.*..***.. ...*. *.......**.**. |
| RN | YGVEAFSDSLRRELTYFGVKVAIIEPGGFKTNVTNMERLSDNLKKLWDQTTEEVKEIYGE |
| HP | QYFDALYNIMKEGLLNCSTNLNLVTDCMEHALTSVHPRTRYSAGWDAKFFFIPLSYLPTS |
| | .. *. . *.. . .**..*.************ *******.*******..*.***** |
| RN | KFQDSYMKAMESLVNTCSGDLSLVTDCMEHALTSCHPRTRYSPGWDAKFFYLPMSYLPTF |
| HP | LADYILTRSWPKPAQAV |
| | *.* .. .. ***.*. |
| RN | LSDAVIHWGSVKPARAL |

Furthermore, the search of GenBank using the base sequence of the present cDNA revealed that there existed some ESTs possessing the homology of 90% or more (for example, Accession No. R35197), but any of them was shorter than the present cDNA and did not contain the initiation codon.

The rat retinol dehydrogenase has been found as a microsomal membrane protein participating in the retinoic acid biosynthesis in the liver [Chai, X. et al., J. Biol. Chem. 270: 28408-28412 (1995)]. Accordingly, its homologue, the protein of the present invention, is considered to possess a similar function and can be utilized for diagnosis and treatment of diseases caused by the abnormality of this protein.

<HP01347> (Sequence Number 3, 21, 39)

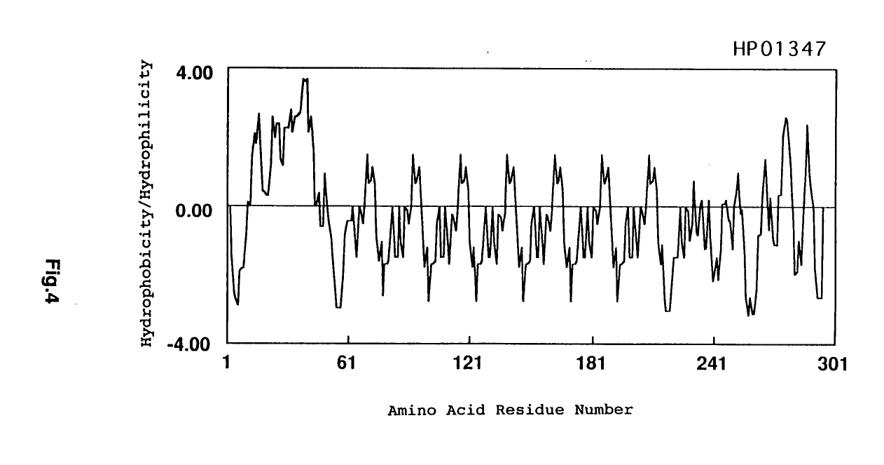

Determination of the whole base sequence for the cDNA insert of clone HP01347 obtained from the human liver cDNA libraries revealed the structure consisting of a 5′-non-translation region of 24 bp, an ORF of 891 bp, and a 3′-non-translation region of 728 bp. The ORF codes for a protein consisting of 296 amino acid residues with one transmembrane domain at the N-terminal. FIG. 4 depicts the hydrophobicity/hydrophilicity profile of the present protein obtained by the Kyte-Doolittle method. It was indicated that the present protein remained in the membrane from the observation that the urokinase secretion was not identified and the urokinase activity was detected on the membrane surface, upon transduction into the COS7 cells of an expression vector in which a HindIII-SacI fragment (treated with the mung-bean nuclease) containing a cDNA fragment encoding the N-terminal 73 amino acid residues in the present protein was inserted at the HindIII-EcoRV site of pSSD3. Therefore, the present protein is considered to be a type-II membrane protein. The in vitro translation resulted in the formation of a translation product of 33 kDa that was almost consistent with the molecular weight of 33,527 predicted from the ORF.

The search of the protein data base using the amino acid sequence of the present protein revealed that the protein was analogous to the human HIV envelope glycoprotein gp120-binding C-type lectin (GenBank Accession No. M98457). Table 6 indicates the comparison of the amino acid sequences between the human protein of the present invention (HP) and the human HIV envelope glycoprotein gp120-binding C-type lectin (CL). - represents a gap, * represents an amino acid residue identical to that in the protein of the present invention, and . represents an amino acid residue analogous to that in the protein of the present invention. The both proteins possessed a homology of 85.6% among 284 amino acid residues. There is observed at the downstream of the transmembrane domain a sequence with seven repetition of Ile-Tyr-Gln-Xaa-Leu-Thr-Xaa-Leu-Lys-Ala-Ala-Val-Gly-Glu-Leu-Xaa-Xaa-Xaa-Ser-Lys-Xaa-Gln-Xaa.

TABLE 6

| | |
|---|---|
| HP | MSDSKEPRVQQLGLL--------------------GCLGHGALVLQLLSFMLLAGVLVAI |
| | ********.****** ******.******** **** . |
| CL | MSDSKEPRLQQLGLLEEEQLRGLGFRQTRGYKSLAGCLGHGPLVLQLLSFTLLAG----L |
| HP | LVQVSKVPSSLSQEQSEQDAIYQNLTQLKAAVGELSEKSKLQEIYQELTQLKAAVGELPE |
| | **********.***** ******************************************* |
| CL | LVQVSKVPSSISQEQSRQDAIYQNLTQLKAAVGELSEKSKLQEIYQELTQLKAAVGELPE |
| HP | KSKLQEIYQELTRLKAAVGELPEKSKLQEIYQELTRLKAAVGELPEKSKLQEIYQELTRL |
| | ***********************************.*************.********** |
| CL | KSKLQEIYQELTRLKAAVGELPEKSKLQEIYQELTWLKAAVGELPEKSKHQEIYQELTRL |
| HP | KAAVGELPEKSKLQEIYQELTELKAAVGELPEKSKLQEIYQELTQLKAAVGELPDQSKQQ |
| | ************ ******** ************* ********.*********..**** |
| CL | KAAVGELPEKSKQQEIYQELTRLKAAVGELPEKSKQQEIYQELTRLKAAVGELPEKSKQQ |
| HP | QIYQELTDLKTAFERLCRHCPKDWTFFQGNCYFMSNSQRNWHDSVTACQEVRAQLVVIKT |
| | .******.**.* ****..** .*********************.***.** *******. |
| CL | EIYQELTQLKAAVERLCHPCPWEWTFFQGNCYFMSNSQRNWHDSITACKEVGAQLVVIKS |
| HP | AEEQLPAVLEQWRTQQ |
| | **** *. *... |
| CL | AEEQNFLQLQSSRSNRFTWMGLSDLNQEGTWQWVDGSPLLPSFKQYWNRGEPNNVGEEDC |

Furthermore, the search of GenBank using the base sequence of the present cDNA revealed that there existed some ESTs possessing the homology of 90% or more (for example, Accession No. H90360), but it can not be assessed whether these ESTs with partial sequences code for the same protein as the protein of the present invention.

The present protein, because of being a type-II membrane protein, is considered to exert its function as a receptor on the membrane surface with the C-terminal side exposed outside the cells or after undergoing a processing followed by being excreted in the serum. Hereupon, the human HIV envelope glycoprotein gp120-binding C-type lectin that is highly homologous with the present protein has been found as a CD4-independent HIV receptor [Curtis, B. M. et al., Proc. Natl. Acad. Sci. USA 89: 8356-8360 (1992)].

<HP01440> (Sequence Number 4, 22, 40)

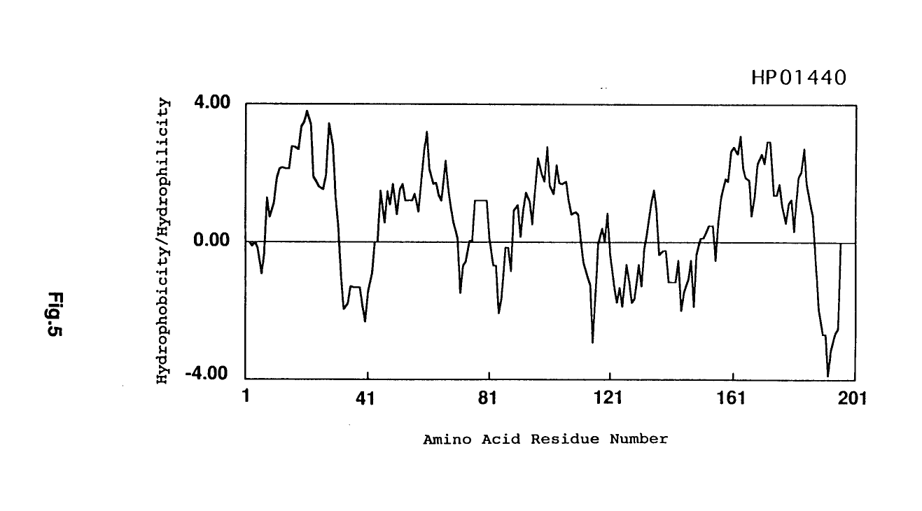

Determination of the whole base sequence for the cDNA insert of clone HP01440 obtained from the human stomach cancer cDNA libraries revealed the structure consisting of a 5′-non-translation region of 37 bp, an ORF of 594 bp, and a 3′-non-translation region of 98 bp. The ORF codes for a protein consisting of 197 amino acid residues with four transmembrane domains. FIG. 5 depicts the hydrophobicity/hydrophilicity profile of the present protein obtained by the Kyte-Doolittle method. The in vitro translation resulted in the formation of a translation product of 21 kDa that was almost consistent with the molecular weight of 20,822 predicted from the ORF.

The search of the protein data base using the amino acid sequence of the present protein revealed that the protein was analogous to the human tumor-associated antigen L6 (SWISS-PROT Accession No. P30408). Table 7 indicates the comparison of the amino acid sequences between the human protein of the present invention (HP) and the human tumor-associated antigen L6 (L6). - represents a gap, * represents an amino acid residue identical to that in the protein of the present invention, and . represents an amino acid residue analogous to that in the protein of the present invention. The both proteins possessed a homology of 47.0% among the entire regions.

TABLE 7

| | |
|---|---|
| HP | MCTGKCARCVGLSLITLCLVCIVANALLLVPNGETSWTNTNHLSLQVWLMGGFIGGGLMV |
| | ** ******.* **..* *.**.** ** *****.....**** **...*..****.. |
| L6 | MCYGKCARCIGHSLVGLALLCIAANILLYFPNGETKYASENHLSRFVWFFSGIVGGGLLM |
| HP | LCPG---IAAVRAGGKGCCGAGCCGNRCRMLRSVFSSAFGVLGAIYCLSVSGAGLRNGPR |
| | * *. *. ... **** . **.** **.**... .*. *. **. *.. ** .** |
| L6 | LLPAFVFIGLEQDDCCGCCGHENCGKRCAMLSSVLAALIGIAGSGYCVIVAALGTAEGPL |
| HP | CLMN-GEWGYHFEDTAGAYLLNRTLWDRCEAPPRVVPWNVTLFSLLVAASCLEIVLCGIQ |
| | ** . *.*.* *..*.*.***. . *. *..* ..* ***.***.*.* . .*..** ** |
| L6 | CLDSLGQWNYTFASTEGQYLLDTSTWSECTEPKHIVEWNVSLFSILLALGGIEFILCLIQ |
| HP | LVNATIGVFCGDCRKKQDTPH |
| | ..*...* .** * ..*. |
| L6 | VINGVLGGICGFCCSHQQQYDC |

Furthermore, the search of GenBank using the base sequence of the present cDNA revealed that there existed some ESTs possessing the homology of 90% or more and also containing the initiation codon (for example, Accession No. T55097), but many sequences were not distinct and the same ORF as that in the present cDNA was not identified.

The human tumor-associated antigen L6 is a member of a membrane antigen TM4 superfamily proteins which are expressed in large quantities on the surface of human tumor cells [Marken, J. S. et al., Proc. Natl. Acad. Sci. USA 89: 3503-3507 (1992)]. Since these membrane antigens are expressed specifically on some specified cells or cancer cells, antibodies against these antigens, if constructed, are useful for a variety of diagnoses and as carriers for the drug delivery. In addition, the cells in which genes of these membrane antigens are transduced and the membrane antigens are expressed are applicable for detection of the corresponding ligands and so on.

<HP01526> (Sequence Number 5, 23, 41)

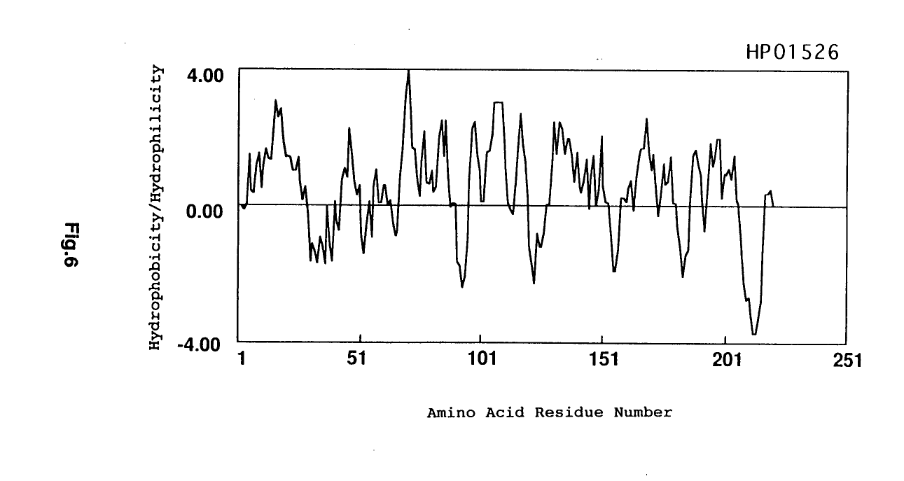

Determination of the whole base sequence for the cDNA insert of clone HP01526 obtained from the human stomach cancer cDNA libraries revealed the structure consisting of a 5′-non-translation region of 83 bp, an ORF of 666 bp, and a 3′-non-translation region of 573 bp. The ORF codes for a protein consisting of 221 amino acid residues with a hydrophobic region of putative six transmembrane domains. FIG. 6 depicts the hydrophobicity/hydrophilicity profile of the present protein obtained by the Kyte-Doolittle method. The in vitro translation resulted in the formation of a translation product of 23 kDa that was almost consistent with the molecular weight of 25,030 predicted from the ORF.

The search of the protein data base using the amino acid sequence of the present protein revealed that the protein was analogous to the mouse interstitial cell protein (GenBank Accession No. X96618). Table 8 indicates the comparison of the amino acid sequences between the human protein of the present invention (HP) and the mouse interstitial cell protein (MM). - represents a gap, * represents an amino acid residue identical to that in the protein of the present invention, and . represents an amino acid residue analogous to that in the protein of the present invention. The both proteins possessed a homology of 79.6% among the entire regions.

TABLE 8

| | |
|---|---|
| HP | MEAGGFLDSLIYGACVVFTLGMFSAGLSDLRHMRMTRSVDNVQFLPFLTTEVNNLGWLSY |
| | ***** **.. .***.*******.********..******.********.****.**** |
| MM | MEAGGVADSFLSSACVLFTLGMFSTGLSDLRHMQRTRSVDNIQFLPFLTTDVNNLSWLSY |
| HP | GALKGDGILIVVNTVGAALQTLYILAYLHYCPRKRVVLLQTATLLGVLLLGYGYFWLLVP |
| | *.*****.**.**.***.************.*.*. *********.************** |
| MM | GVLKGDGTLIIVNSVGAVLQTLYILAYLHYSPQKHGVLLQTATLLAVLLLGYGYFWLLVP |
| HP | NPEARLQQLGLFCSVFTISMYLSPLADLAKVIQTKSTQCLSYPLTIATLLTSASWCLYGF |
| | . ****************************..****** **..******. ****..*** |
| MM | DLEARLQQLGLFCSVFTISMYLSPLADLAKIVQTKSTQRLSFSLTIATLFCSASWSIYGF |
| HP | RLRDPYIMVSNFPGIVTSFIRFWLFWKYPQEQDRNYWLLQT |
| | ******* *.*.***.**.**. ** ***.****.*.**** |
| MM | RLRDPYIAVPNLPGILTSLIRLGLFCKYPPEQDRKYRLLQT |

Furthermore, the search of GenBank using the base sequence of the present cDNA revealed that there existed some ESTs possessing the homology of 90% or more and also containing the initiation codon (for example, Accession No. H02682), but many sequences were not distinct and the same ORF as that in the present cDNA was not identified.

The mouse interstitial cell protein has been cloned as a membrane protein that is expressed with highly increasing in interstitial cells stimulated by a cytokine [Tagoh, H. et al., Biochem. Biophys. Res. Commun. 221: 744-749 (1996)]. Since these membrane proteins are expressed specifically on some specified cells and cancer cells, antibodies against these proteins, if constructed, are useful for a variety of diagnoses and as carriers for the drug delivery. In addition, the cells in which genes of these membrane antigens are transduced and the membrane antigens are expressed are applicable for detection of the corresponding ligands and so on.

<HP10230> (Sequence Number 6, 24, 42)

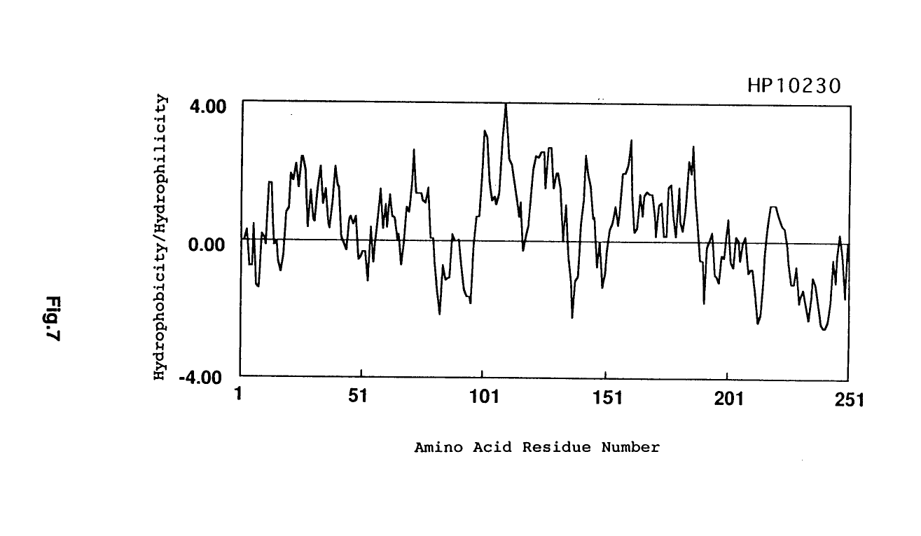

Determination of the whole base sequence for the cDNA insert of clone HP10230 obtained from the human stomach cancer cDNA libraries revealed the structure consisting of a 5′-non-translation region of 190 bp, an ORF of 756 bp, and a 3′-non-translation region of 2099 bp. The ORF codes for a protein consisting of 251 amino acid residues with at least one transmembrane domain. FIG. 7 depicts the hydrophobicity/hydrophilicity profile of the present protein obtained by the Kyte-Doolittle method. The in vitro translation resulted in the formation of a translation product of 30 kDa that was almost consistent with the molecular weight of 28,800 predicted from the ORF.

The search of the protein data base using the amino acid sequence of the present protein revealed that the protein was analogous to the nematode hypothetical protein F25D7.1 (GenBank Accession No. Z78418). Table 9 indicates the comparison of the amino acid sequences between the human protein of the present invention (HP) and the nematode hypothetical protein F25D7.1 (CE). - represents a gap, * represents an amino acid residue identical to that in the protein of the present invention, and represents an amino acid residue analogous to that in the protein of the present invention. The both proteins possessed a homology of 49.8% among the entire regions.

TABLE 9

| | |
|---|---|
| HS | MSDIGDWFRSIPAITRYWFAATVAVPLVGKLGLISPAYLFL-WPEAFLYRFQTWRPITAT |
| | *..... .** .***** *.. .**.*..*.*. ...** * . . .**.***.** |
| CE | MDLENFLLGIPIVTRYWFLASTIIPLLGRFGFINVQWMFLQW-DLVVNKFQFWRPLTAL |
| HS | FYFPVGPGTGFLYLVNLYFLYQYSTRLETGAFDGRPADYLFMLLFNW-ICIVITGLAMDM |
| | .*.**.* *** .*. ****.**. **.... **.*******.*** .* . .*.*. |
| CE | IYYPVTPQTGFHWLMMCYFLYNYSKALESETYRGRSADYLFMLIFNWFFCSGLC-MALDI |
| HS | QLLMIPLIMSVLYVWAQLNRDMIVSFWFGTRFKACYLPWVILGFNYIIGGSVINELIGNL |
| | .*. *...****** *.*.* ******* ** * *****. *** .. *. .***.* * |
| CE | YFLLEPMVISVLYVWCQVNKDTIVSFWFGMRFPARYLPWVLWGFNAVLRGGGTNELVGIL |
| HS | VGHLYFFLMFRYPMDLGGRNFLSTPQFLYRWLPSRRGGVSGFGVPPASMRRAADQNGGGG |
| | *** ***. ..** . * ...***.**.* .*. **. * * * * * |
| CE | VGHAYFFVALKYPDEYGV-DLISTPEFLHRLIPDEDGGIHG---QDGNIRGARQQPRG-- |
| HS | RHNW--GQGFRLGDQ |
| | * * * * *** |
| CE | -HQWPGGVGARLGGN |

Furthermore, the search of GenBank using the base sequence of the present cDNA revealed that there existed some ESTs possessing the homology of 90% or more and also containing the initiation codon (for example, Accession No. WO1493), but many sequences were not distinct and the same ORF as that in the present cDNA was not identified.

<HP10389> (Sequence Number 7, 25, 43)

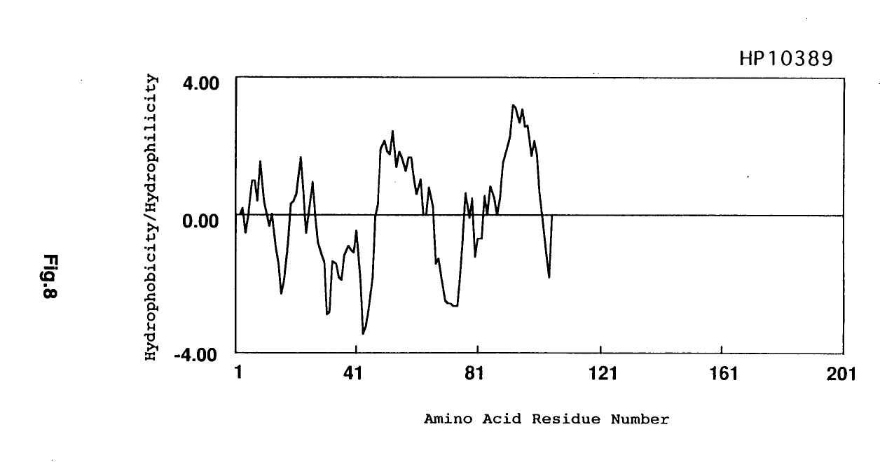

Determination of the whole base sequence for the cDNA insert of clone HP10389 obtained from the human epidermoid carcinoma cell line KBc cDNA libraries revealed the structure consisting of a 5′-non-translation region of 62 bp, an ORF of 321 bp, and a 3′-non-translation region of 270 bp. The ORF codes for a protein consisting of 106 amino acid residues with a hydrophobic region of putative two transmembrane domains. FIG. 8 depicts the hydrophobicity/hydrophilicity profile of the present protein obtained by the Kyte-Doolittle method. The in vitro translation resulted in the formation of a translation product of 12 kDa that was almost consistent with the molecular weight of 11,528 predicted from the ORF.

The search of the protein data base using the amino acid sequence of the present protein revealed that the protein was not analogous to any of known proteins. Furthermore, the search of GenBank using the base sequence of the present cDNA revealed that there existed some ESTs possessing the homology of 90% or more (for example, Accession No. H70816), but many sequences were not distinct and the same ORF as that in the present cDNA was not identified.

<HP10408> (Sequence Number 8, 26, 44)

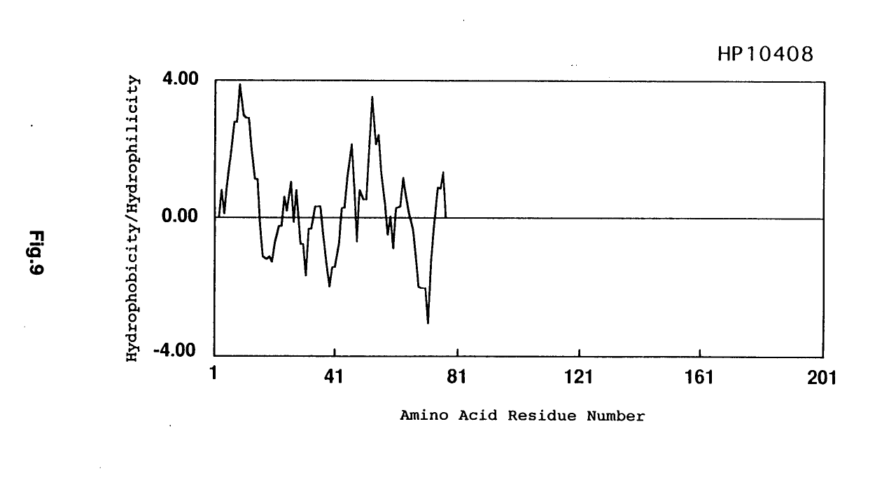

Determination of the whole base sequence for the cDNA insert of clone HP10408 obtained from the human stomach cancer cDNA libraries revealed the structure consisting of a 5′-non-translation region of 74 bp, an ORF of 237 bp, and a 3′-non-translation region of 128 bp. The ORF codes for a protein consisting of 78 amino acid residues with a putative signal sequence at the N-terminal as well as a sequence of one putative interior transmembrane domain. FIG. 9 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-BglII fragment (after the Klenow treatment) containing a cDNA fragment encoding the N-terminal 70 amino acid residues in the present protein was inserted at the HindIII-EcoRV site of pSSD3. The in vitro translation resulted in the formation of a translation product of 9 kDa that was almost consistent with the molecular weight of 8,396 predicted from the ORF.

Furthermore, the search of GenBank using the base sequence of the present cDNA revealed that there existed some ESTs possessing the homology of 90% or more (for example, Accession No. T94049), but they were shorter than the present cDNA and any molecule containing the initiation codon was not identified.

<HP10412> (Sequence Number 9, 27, 45)

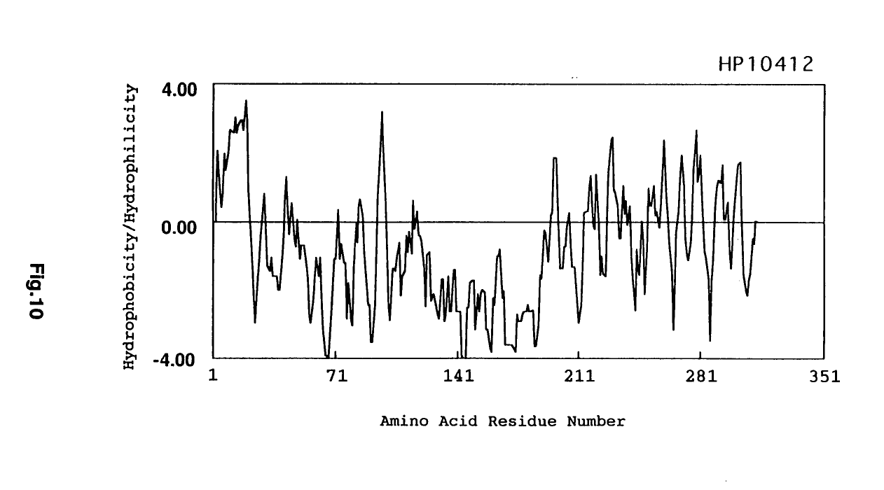

Determination of the whole base sequence for the cDNA insert of clone HP10412 obtained from the human stomach cancer cDNA libraries revealed the structure consisting of a 5′-non-translation region of 55 bp, an ORF of 945 bp, and a 3′-non-translation region of 131 bp. The ORF codes for a protein consisting of 314 amino acid residues with one transmembrane domain at the N-terminal. FIG. 10 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-ApaI fragment (treated with mung-bean nuclease) containing a cDNA fragment encoding the N-terminal 65 amino acid residues in the present protein was inserted at the HindIII-EcoRV site of pSSD3. The in vitro translation resulted in the formation of a translation product of 44 kDa that was somewhat larger than the molecular weight of 35,610 predicted from the ORF.

The search of the protein data base using the amino acid sequence of the present protein revealed that the protein was analogous to the nematode hypothetical protein of 28.5 kDa (SWISS-PROT Accession No. P34623). Table 10 indicates the comparison of the amino acid sequences between the human protein of the present invention (HP) and the nematode hypothetical protein of 28.5 kDa (CE). - represents a gap, * represents an amino acid residue identical to that in the protein of the present invention, and . represents an amino acid residue analogous to that in the protein of the present invention. The both proteins possessed a homology of 42.8% in the C-terminal region of 243 amino acid residues.

TABLE 10

| | |
|---|---|
| HP | MVAPVWYLVAAALLVGFILFLTRSRGRAASAGQEPLHNEELAGAGRVAQPGPLEPEEPRA |
| HP | GGRPRRRRDLGSRLQAQRRAQRVAWAEA--DENEEEAVILAQEEEGVEKPAETHLSGKIG |
| | * .*.**..* . ... .. ... ... ..*.*. . .. . *** |
| CE | MRRNARRRVNRDEQEDGFVNHMMNDGEDVEDLDGGAEQFEYDEDGKKIG |
| HP | AKKLRKLEEKQARKAQREAEEAEREERKRLESQREAEWKKEEERLRLEEEQKEEEE--RK |
| | .* **..*..... ** * ******* *..* * *..*** . *...*.*** ** |
| CE | KRKAAKLQAKEEKRQMREYEVREREERKRREEER--EKKRDEERAKEEADEKAEEERLRK |
| HP | AREEQAQREHEEYLKLKEAFVVEEEGVGETMTEEQSQSFLTEFINYIKQSKVVLLEDLAS |
| | .***....****** .*..*..**** ......*...... .*..*.* .*** ...*.* |
| CE | EREEKERKEHEEYLAMKASFAIEEEG-TDAIEGEEAENLIRDFVDYVKTNKVVNIDELSS |
| HP | QVGLRTQDTINRIQDLLAEGTITGVIDDRGKFIYITPEELAAVANFIRQRGRVSIAELAQ |
| | . **...*..**.*....** . **.*********. **.****.**.******* *.*. |
| CE | HFGLKSEDAVNRLQHFIEEGLVQGVMDDRGKFIYISDEEFAAVAKFINQRGRVSIHEIAE |
| HP | ASNSLIAWGRESPAQAPA |
| | .**.** . *.*. |
| CE | QSNRLIRLETPSAAE |

Furthermore, the search of GenBank using the base sequence of the present cDNA revealed that there existed some ESTs possessing the homology of 90% or more (for example, Accession No. T09311), but it can not be assessed whether these ESTs with partial sequences code for the same protein as the protein of the present invention.

<HP10413> (Sequence Number 10, 28, 46)

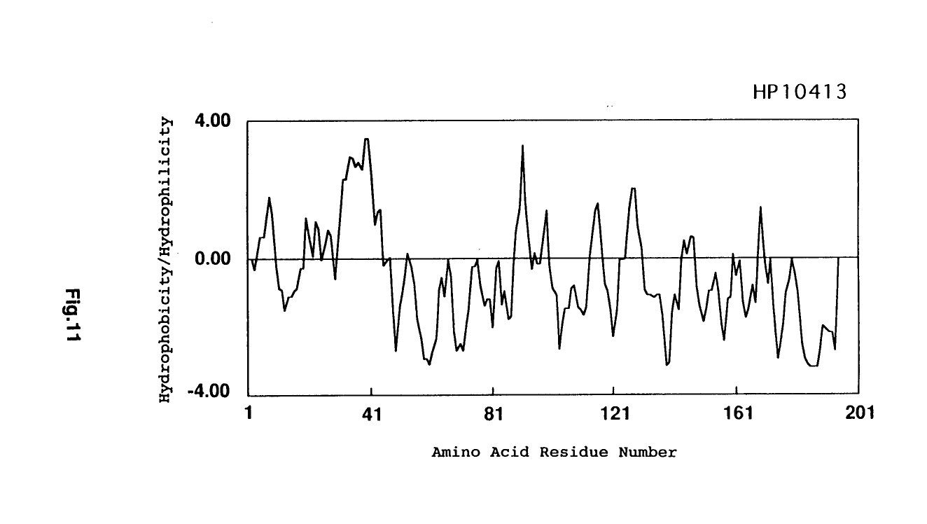

Determination of the whole base sequence for the cDNA insert of clone HP10413 obtained from the human stomach cancer cDNA libraries revealed the structure consisting of a 5′-non-translation region of 78 bp, an ORF of 588 bp, and a 3′-non-translation region of 1209 bp. The ORF codes for a protein consisting of 195 amino acid residues with one transmembrane domain at the N-terminal. FIG. 11 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-PmaCI fragment containing a cDNA fragment encoding the N-terminal 65 amino acid residues in the present protein was inserted at the HindIII-PmaCI site of pSSD3. The in vitro translation resulted in the formation of a translation product of 28 kDa that was somewhat larger than the molecular weight of 21,671 predicted from the ORF.

The search of the protein data base using the amino acid sequence of the present protein revealed that the protein was analogous to the swine steroidal membrane-binding protein (GenBank Accession No. X99714). Table 11 indicates the comparison of the amino acid sequences between the human protein of the present invention (HP) and the swine steroidal membrane-binding protein (SS). - represents a gap, * represents an amino acid residue identical to that in the protein of the present invention, and . represents an amino acid residue analogous to that in the protein of the present invention. The both proteins possessed a homology of 96.4% among the entire regions.

TABLE 11

| | |
|---|---|
| HP | MAAEDVVATGADPSDLESGGLLHEIFTSPLNLLLLGLCIFLLYKIVRGDQPAASGDSDDD |
| | ******.*******.**.************************************ ***** |
| SS | MAAEDVAATGADPSELEGGGLLHEIFTSPLNLLLLGLCIFLLYKIVRGDQPAAS-DSDDD |
| HP | EPPPLPRLKRRDFTPAELRRFDGVQDPRILMAINGKVFDVTKGRKFYGPEGPYGVFAGRD |
| | ************************************************************ |
| SS | EPPPLPRLKRRDFTPAELRRFDGVQDPRILMAINGKVFDVTKGRKFYGPEGPYGVFAGRD |
| HP | ASRGLATFCLDKEALKDEYDDLSDLTAAQQETLSDWESQFTFKYHHVGKLLKEGEEPTVY |
| | **************************.******.**.*********************** |
| SS | ASRGLATFCLDKEALKDEYDDLSDLTPAQQETLNDWDSQFTFKYHHVGKLLKEGEEPTVY |
| HP | SDEEEPKDESARKND |
| | *************** |
| SS | SDEEEPKDESARKND |

Furthermore, the search of GenBank using the base sequence of the present cDNA revealed that there existed some ESTs possessing the homology of 90% or more (for example, Accession No. AA021062), but many sequences were not distinct and the same ORF as that in the present cDNA was not identified.

<HP10415> (Sequence Number 11, 29, 47)

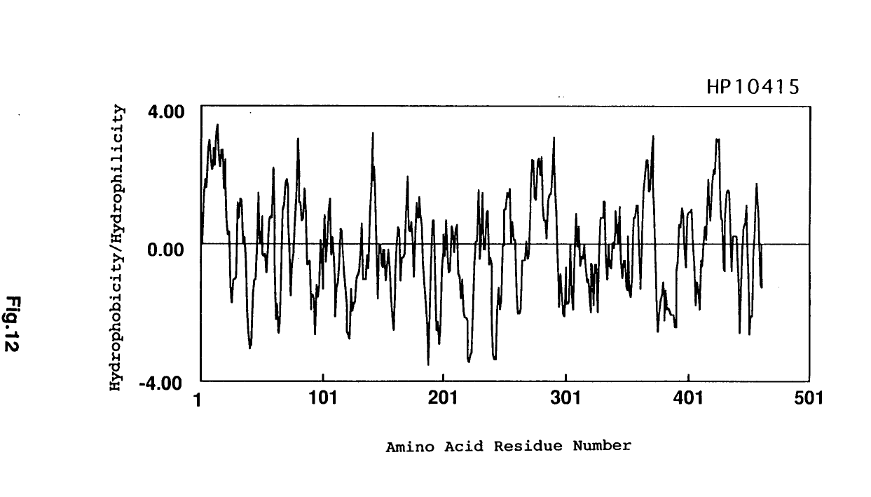

Determination of the whole base sequence for the cDNA insert of clone HP10415 obtained from the human stomach cancer cDNA libraries revealed the structure consisting of a 5′-non-translation region of 71 bp, an ORF of 1389 bp, and a 3′-non-translation region of 103 bp. The ORF codes for a protein consisting of 462 amino acid residues with one transmembrane domain at the N-terminal. FIG. 12 depicts the hydrophobicity/hydrophilicity profile of the present protein obtained by the Kyte-Doolittle method. The in vitro translation resulted in the formation of a translation product of 48 kDa that was somewhat smaller than the molecular weight of 52,458 predicted from the ORF.

The search of the protein data base using the amino acid sequence of the present protein revealed that the protein was analogous to the cytochrome P450 as exemplified by the simian cytochrome P450IIIA8 (SWISS-PROT Accession No. P33268). Table 12 indicates the comparison of the amino acid sequences between the human protein of the present invention (HP) and the simian cytochrome P450IIIA8 (CP). - represents a gap, * represents an amino acid residue identical to that in the protein of the present invention, and . represents an amino acid residue analogous to that in the protein of the present invention. The both proteins possessed a homology of 21.3% among the entire regions.

TABLE 12

| | |
|---|---|
| HP | MLDFAIFAVTFLLALVGAVLYLYPASRQAAGIPGITPTEEKDGNLPDIVN-SGSLHEF |
| | .***.. ..**** . ... .*. . * .*.. . .. .* |
| CP | MDLIPDLAVETWLLLAVTLVLLYLYGTHSHGLFKKLGIPGPTPLPLLGNILSYRKGFWTF |
| HP | LVNLHERYGPVVSFWFGRRLVVSLGTVDVLKQHINPNKTLDPFETMLK-SLLRYQSGGGS |
| | .. ...** * .*. **. *..... ...* . .. . *... . . . ... * |
| CP | DMECYKKYGKVWGFYDGRQPVLAITDPNMIK-TVLVKECYSVFTNRRPFGPVGFMKNAIS |
| HP | VSEN----HMRKKLYENGVTDSLKSNFALLLKLSEELLDKWLSYPET-QHVPLSQHMLGF |
| | ..*. ..*. * . ....**. ... * .. *. . . .** ..*.*.. . .. |
| CP | IAEDEEWKRIRSLLSPTFTSGKLKEMVPILAKYGDVLVRNLRREAETGKPVTLKDVFGAY |
| HP | AMKSVTQMVMG----------STF-EDDQEVIRFQKNHGTVWSEIGKGFLDGSLD--KNM |
| | .*. .* .* ..* *......**. . * . *. *. . |
| CP | SMDVITSTSFGVNIDSLNNPQDPFVENTKKLLRFDFLDPFFLSITIFPFIIPILEVLNIS |
| HP | TRKKQYEDALMQ-LESVLRNIIKE-RKGR-NFSQHIF-----IDSLVQGNLNDQQILEDS |
| | . .. .. *.. .... . .*. .* * .* * .. .. . .*.* .....* |
| CP | IFPREVTSFLRKSVKRIKESRLKDTQKHRVDFLQLMIDSQNSKETESHKALSDLELVAQS |
| HP | MIFSLASCIITAKLCTWAICFLTTSEEVQKKLYEEINQVF-GNGPVTPEKIEQLRYCQHV |
| | .** .*.. .*... .. *. *.* .**.** ***. *. ...* * ... *. * . * |
| CP | IIFIFAGYETTSSVLSFIIYELATHPDVQQKLQEEIDTVLPNKAPPTYDTVLQMEYLDMV |
| HP | LCETVRTAKLTPVSAQLQDIEGKIDRFIIPRETLVLYALGVVLQDPNTWPSPHKFDPDRF |
| | . **.*. .. ... . . .*. ..**....*. . .. .**. **.*.** *.** |
| HP | VNETLRIFPIAMRLERVCKKDVEINGIFIPKGVVVMIPSYALHHDPKYWPEPKFLPERF |
| HP | ----DDELVMKTFSSLGFSGTQECPELRFAYMVTTVLLSVLVKRLHLLSVEGQVIETKYE |
| | .*.. .....* **...* ..*** * .. . ..... . . .. * * |
| CP | SKKNNDNIDPYIYTPFG-SGPRNCIGMRFALMNMKLAIIRVLQNFSFKPCKETQIPLKLR |
| HP | LVTSSREEAWITVSKRY |
| | * |
| CP | LGGLLQTEKPIVLKIESRDGTVSGA |

Furthermore, the search of GenBank using the base sequence of the present cDNA revealed that there existed some ESTs possessing the homology of 90% or more (for example, Accession No. AA381169), but it can not be assessed whether these ESTS with partial sequences code for the same protein as the protein of the present invention.

The cytochrome P450 participates in the drug metabolism and can be utilized as a catalyst in organic synthesis reactions such as oxidation and so on.

<HP10419> (Sequence Number 12, 30, 48)

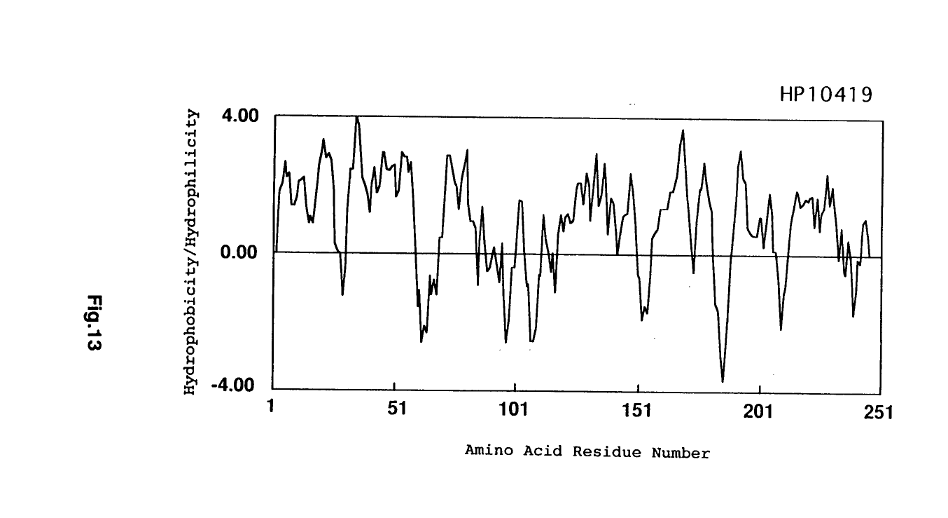

Determination of the whole base sequence for the cDNA insert of clone HP10419 obtained from the human stomach cancer cDNA libraries revealed the structure consisting of a 5′-non-translation region of 170 bp, an ORF of 744 bp, and a 3′-non-translation region of 1116 bp. The ORF codes for a protein consisting of 247 amino acid residues with a hydrophobic region of putative seven transmembrane domains. FIG. 13 depicts the hydrophobicity/hydrophilicity profile of the present protein obtained by the Kyte-Doolittle method.

The search of GenBank using the base sequence of the present cDNA revealed that there existed some ESTs possessing the homology of 90% or more (for example, Accession No. AA340663), but it can not be assessed whether these ESTs with partial sequences code for the same protein as the protein of the present invention.

<HP10424> (Sequence Number 13, 31, 49)

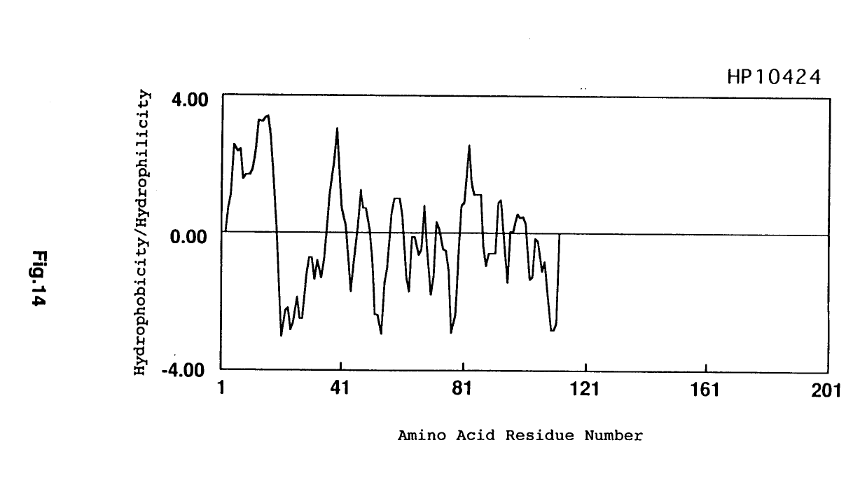

Determination of the whole base sequence for the cDNA insert of clone HP10424 obtained from the human stomach cancer cDNA libraries revealed the structure consisting of a 5′-non-translation region of 97 bp, an ORF of 342 bp, and a 3′-non-translation region of 54 bp. The ORF codes for a protein consisting of 113 amino acid residues with one transmembrane domain at the N-terminal. FIG. 14 depicts the hydrophobicity/hydrophilicity profile of the present, protein obtained by the Kyte-Doolittle method. It was indicated that the present protein remained in the membrane from the observation that the urokinase secretion was not identified upon transduction into the COS7 cells of an expression vector in which a HindIII-AccI fragment (after the Klenow treatment) containing a cDNA fragment encoding the N-terminal 58 amino acid residues in the present protein was inserted at the HindIII-SmaI site of pSSD3. The in vitro translation resulted in the formation of a translation product of 14 kDa that was somewhat larger than the molecular weight of 12,784 predicted from the ORF.

Furthermore, the search of GenBank using the base sequence of the present cDNA revealed that there existed some ESTs possessing the homology of 90% or more (for example, Accession No. AA401979), but it can not be assessed whether these ESTs with partial sequences code for the same protein as the protein of the present invention.

<HP10428> (Sequence Number 14, 32, 50)

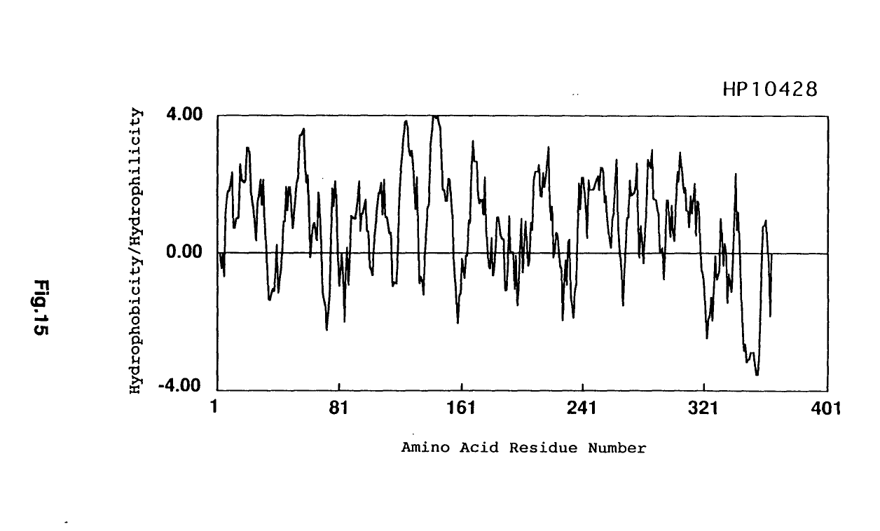

Determination of the whole base sequence for the cDNA insert of clone HP10428 obtained from the human epidermoid carcinoma cell line KBc cDNA libraries revealed the structure consisting of a 5′-non-translation region of 287 bp, an ORF of 1098 bp, and a 3′-non-translation region of 659 bp. The ORF codes for a protein consisting of 365 amino acid residues with a hydrophobic region of putative nine transmembrane domains. FIG. 15 depicts the hydrophobicity/hydrophilicity profile of the present protein obtained by the Kyte-Doolittle method. The result of the in vitro translation did not reveal the formation of distinct bands and only revealed the formation of smeary bands at the high-molecular-weight position.

The search of the protein data base using the amino acid sequence of the present protein revealed that the protein was analogous to the baker's yeast hypothetical membrane protein YML038c (NBRF Accession No. S49741). Table 13 indicates the comparison of the amino acid sequences between the human protein of the present invention (HP) and the baker's yeast hypothetical membrane protein YML038c (SC). - represents a gap, * represents an amino acid residue identical to that in the protein of the present invention, and . represents an amino acid residue analogous to that in the protein of the present invention. The both proteins possessed a homology of 26.3% among the N-terminal region of 281 amino acid residues.

TABLE 13

| | |
|---|---|
| HP | MGRWALDVAFLWKAVLTLGLVL-LYYCFSIGITFYNKWL-----TKSFHFPLFMTMLHLA |
| | *..*. *.* *....**.*. . .. .*...* .* * |
| SC | MNRTVFLAFVFGWYFCS-IALSIYNRWMFDPKDGLGIGYPVLVTTFHQA |
| HP | VIFLFSALSRALVQ---CSSHRARVVLSWADYLRRVAPTALATALDVGLSNWSFLYVTVS |
| | ...*.*.. * . . * . ..*. .*. . ***.*.* *.**** ** **... |
| SC | TLWLLSGIYIKLRHKPVKNVLRKNNGFWSFFLKFLLPTAVASAGDIGLSNVSFQYVPLT |
| HP | LYTMTKSSAVLFILIFSLIFKLEEL--RAALVLVVLLIAGGLFMF------TYKSTQ-FN |
| | .**..***.. *.*.*. *****.. . ** ....... .*..* . *..* . |
| SC | IYTIIKSSSIAFVLLFGCIFKLEKFHWKLALSVIIMFVGVALMVFKPSDSTSTKNDQALV |
| HP | VEGFALVLGASFIGGIRWRLTQMLLQKAELGLQNPIDTMFHLQPLMFLGLFPLFAVFEGL |
| | . * ***..* ..*.**. **..*... . ... .. . .* . . . |
| SC | IFGSFLVLASSCLSGLRWVYTQLMLRNNPIQTNTAAAVEES-DGALFTENEDNVDNEPVV |
| HP | HLSTSEKIFRFQDT-GLLLRVLGSLFLGGILAFGLGFSEFLLVSRTSSLTLSIAGIFKEV |
| | .*...... .* .. .... *. *....* ... ***... . ..* ..**. |
| SC | NLANNKMLENFGESKPHPIHTIHQ--LAPIMGITLLLTS-LLVEKPFPGIFS-SSIFRLD |
| HP | CTLLLAAHLLGDQISLLNWLGFALCLSGISLHVALKALHSRGDGGPKALKGLGSSPDLEL |
| SC | TSNGGVGTETTVLSIVRGIVLLILPGFAVFLLTICEFSILEQTPVLTVSIVGIVKELLTV |
| HP | LLRSSQREEGDNEEEEYFVAQGQQ |
| SC | IFGIIILSERLSGFYNWLGMLIIMADVCYYNYFRYKQDLLQKYHSVSTQDNRNELKGFQD |

Furthermore, the search of GenBank using the base sequence of the present cDNA revealed that there existed some ESTs possessing the homology of 90% or more (for example, Accession No. AA018345), but it can not be assessed whether these ESTs with partial sequences code for the same protein as the protein of the present invention.

<HP10429> (Sequence Number 15, 33, 51)

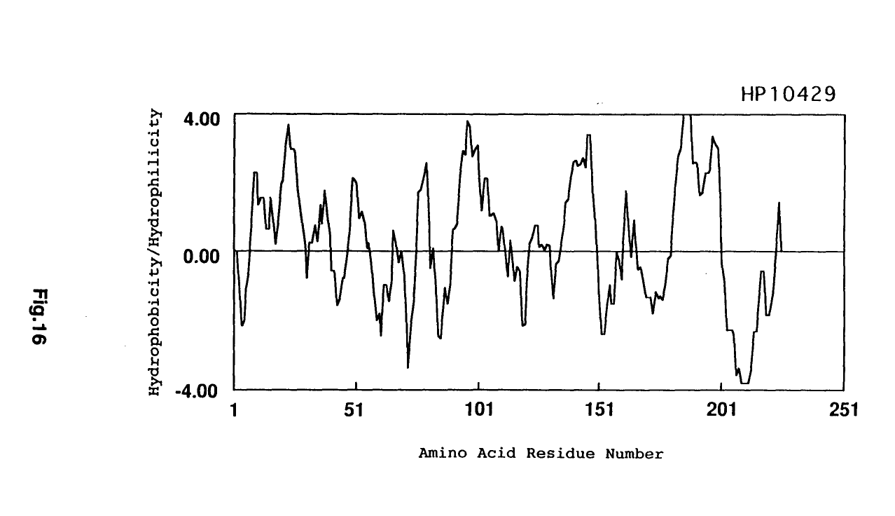

Determination of the whole base sequence for the cDNA insert of clone HP10429 obtained from the human stomach cancer cDNA libraries revealed the structure consisting of a 5′-non-translation region of 156 bp, an ORF of 681 bp, and a 3′-non-translation region of 206 bp. The ORF codes for a protein consisting of 226 amino acid residues with four transmembrane domains. FIG. 16 depicts the hydrophobicity/hydrophilicity profile of the present protein obtained by the Kyte-Doolittle method. The in vitro translation resulted in the formation of a translation product of 25 kDa that was almost consistent with the molecular weight of 25,321 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. AA315933), but it can not be assessed whether these ESTs with partial sequences code for the same protein as the protein of the present invention.

<HP10432> (Sequence Number 16, 34, 52)

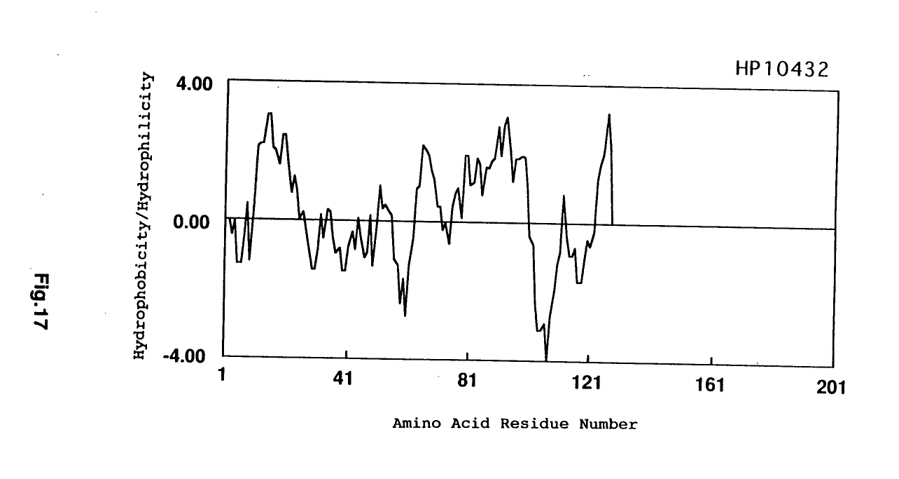

Determination of the whole base sequence for the cDNA insert of clone HP10429 obtained from the human liver cDNA libraries revealed the structure consisting of a 5′-non-translation region of 28 bp, an ORF of 390 bp, and a 3′-non-translation region of 554 bp. The ORF codes for a protein consisting of 129 amino acid residues with a signal-like sequence at the N-terminal and one interior transmembrane domain. Therefore, the present protein is considered to be a type-I membrane protein. FIG. 17 depicts the hydrophobicity/hydrophilicity profile of the present protein obtained by the Kyte-Doolittle method.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. T74424), but the same ORF as that in the present cDNA was not identified.

<HP10433> (Sequence Number 17, 35, 53)

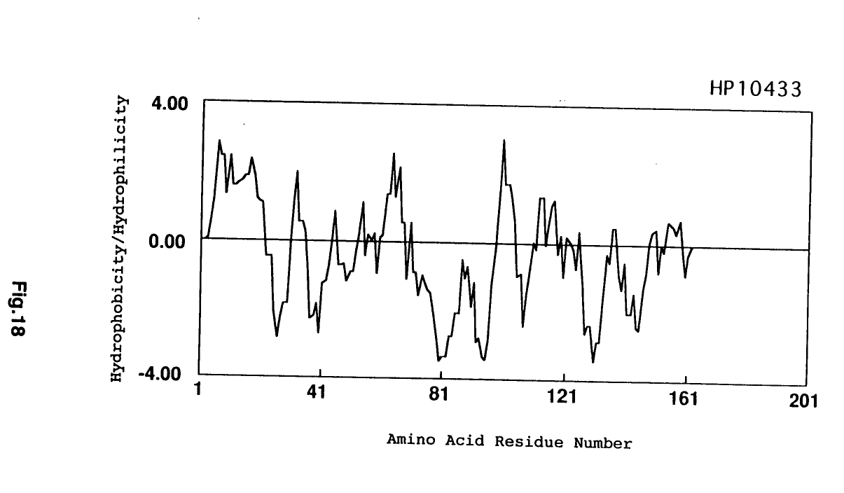

Determination of the whole base sequence for the cDNA insert of clone HP10433 obtained from the human liver cDNA libraries revealed the structure consisting of a 5′-non-translation region of 72 bp, an ORF of 492 bp, and a 3′-non-translation region of 131 bp. The ORF codes for a protein consisting of 163 amino acid residues with one transmembrane domain at the N-terminal. FIG. 18 depicts the hydrophobicity/hydrophilicity profile of the present protein obtained by the Kyte-Doolittle method. It was indicated that the present protein remained in the membrane from the observation that the urokinase secretion was not identified upon transduction into the COS7 cells of an expression vector in which a HindIII-Eco81I fragment (treated with the mung-bean nuclease) containing a cDNA fragment encoding the N-terminal 137 amino acid residues in the present protein was inserted at the HindIII-EcoRV site of pSSD3. Therefore, the present protein is considered to be a type-II membrane protein. The in vitro translation resulted in the formation of a translation product of 21 kDa that was almost consistent with the molecular weight of 18,617 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. H84693), but many sequences are not distinct and the same ORF as that in the present cDNA was not identified.

<HP10480> (Sequence Number 18, 36, 54)

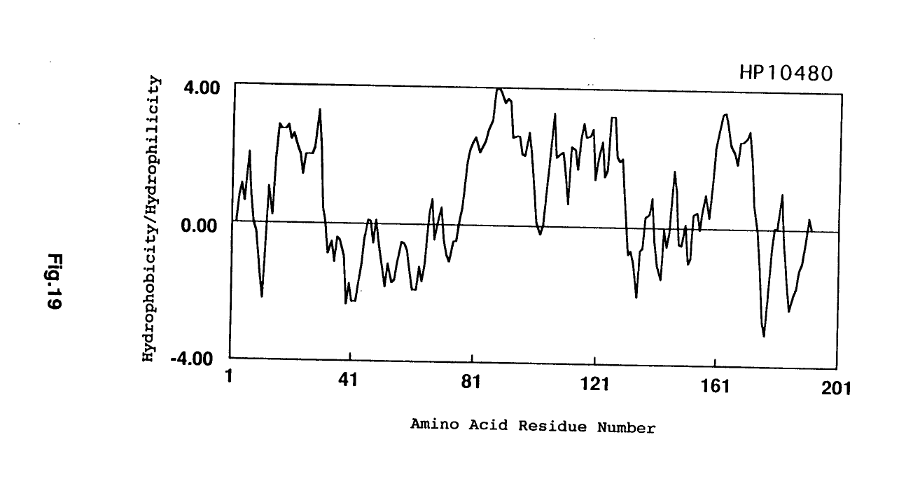

Determination of the whole base sequence for the cDNA insert of clone HP10480 obtained from the human stomach cancer cDNA libraries revealed the structure consisting of a 5′-non-translation region of 79 bp, an ORF of 582 bp, and a 3′-non-translation region of 1253 bp. The ORF codes for a protein consisting of 193 amino acid residues with four transmembrane domains. FIG. 19 depicts the hydrophobicity/hydrophilicity profile of the present protein obtained by the Kyte-Doolittle method. The in vitro translation resulted in the formation of a translation product of 23 kDa that was somewhat larger than the molecular weight of 21,445 predicted from the ORF.

The search of the protein data base using the amino acid sequence of the present protein revealed that the protein was not analogous to any known proteins. Furthermore, the search of GenBank using the base sequence of the present cDNA revealed that there existed some ESTs possessing the homology of 90% or more (for example, Accession No. W93606), but many sequences are not distinct and the same ORF as that in the present cDNA was not identified.

The present invention provides human proteins having transmembrane domains and cDNAs encoding said proteins. All of the proteins of the present invention are putative proteins controlling the proliferation and differentiation of the cells, because said proteins exist on the cell membrane. Therefore, the proteins of the present invention can be used as pharmaceuticals or as antigens for preparing antibodies against said proteins. Furthermore, said DNAs can be used for the expression of large amounts of said proteins. The cells expressing large amounts of membrane proteins with transfection of these membrane protein genes can be applied to the detection of the corresponding ligands, the screening of novel low-molecular medicines, and so on.

In addition to the activities and uses described above, the polynucleotides and proteins of the present invention may exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e. a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J. E. e. a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e. a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6-Nordan, R. In Current Protocols in Immunology. J. E. e. a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11- Bennett, F., Giannotti, J., Clark, S. C. and Turner, K. J. In Current Protocols in Immunology. J. E. e. a. Coligan eds. Vol 1 pp. 6.15.1 John Wiley and Sons, Toronto. 1991; Measurement of mouse and human Interleukin 9- Ciarletta, A., Giannotti, J., Clark, S. C. and Turner, K. J. In Current Protocols in Immunology. J. E. e. a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as , for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I a chain protein and O₂ microglobulin protein or an MHC class IIa chain protein and an MHC class IIβ chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann-et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e. a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, H I and Rovee, DT, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of ytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

1. A protein comprising an amino acid sequence selected from the group consisting of the amino acid sequences of SEQ ID NOS: 1 to 18.

2. A DNA encoding the protein according to claim 1.

3. A cDNA comprising a nucleotide sequence selected from the group consisting of the nucleotide sequences of SEQ ID NOS: 19 to 36.

4. A cDNA according to claim 3, which comprises a nucleotide sequence selected from the group consisting of the nucleotide sequences of SEQ ID NOS: 37 to 54.

5. An expression vector capable of in vitro translating the DNA according to any of claims 2 to 4 or expressing said DNA in an eukaryotic cell.

6. A transformed eukaryotic cell capable of expressing the DNA according to any of claims 2 to 4 to produce the protein according to claim 1.


SEQUENCE LISTING (67 sequences)

SEQ ID NO: 1 - protein, 382 aa, Homo sapiens
MGLLLPLALCILVLCCGAMSPPQLALNPSALLSRGCNDSDVLAVAGFALRDINKDRKDGYVLRLNRVNDAQEYRRGGLGSLFYLTLDVLETDCHVLRKKAWQDCGMRIFFESVYGQCKAIFYMNNPSRVLYLAAYNCTLRPVSKKKIYMTCPDCPSSIPTDSSNHQVLEAATESLAKYNNENTSKQYSLFKVTRASSQWVVGPSYFVEYLIKESPCTKSQASSCSLQSSDSVPVGLCKGSLTRTHWEKFVSVTCDFFESQAPATGSENSAVNQKPTNLPKVEESQQKNTPPTDSPSKAGPRGSVQYLPDLDDKNSQEKGPQEAFPVHLDLTTNPQGETLDISFLFLEPMEEKLVVLPFPKEKARTAECPGPAQNASPLVLPP

SEQ ID NO: 2 - protein, 317 aa, Homo sapiens
MWLYLAAFVGLYYLLHWYRERQVVSHLQDKYVFITGCDSGFGNLLARQLDARGLRVLAACLTEKGAEQLRGQTSDRLETVTLDVTKMESIAAATQWVKEHVGDRGLWGLVNNAGILTPITLCEWLNTEDSMNMLKVNLIGVIQVTLSMLPLVRRARGRIVNVSSILGRVAFFVGGYCVSKYGVEAFSDILRREIQHFGVKISIVEPGYFRTGMTNMTQSLERMKQSWKEAPKHIKETYGQQYFDALYNIMKEGLLNCSTNLNLVTDCMEHALTSVHPRTRYSAGWDAKFFFIPLSYLPTSLADYILTRSWPKPAQAV

SEQ ID NO: 3 - protein, 296 aa, Homo sapiens
MSDSKEPRVQQLGLLGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQLPAVLEQWRTQQ

SEQ ID NO: 4 - protein, 197 aa, Homo sapiens
MCTGKCARCVGLSLITLCLVCIVANALLLVPNGETSWTNTNHLSLQVWLMGGFIGGGLMVLCPGIAAVRAGGKGCCGAGCCGNRCRMLRSVFSSAFGVLGAIYCLSVSGAGLRNGPRCLMNGEWGYHFEDTAGAYLLNRTLWDRCEAPPRVVPWNVTLFSLLVAASCLEIVLCGIQLVNATIGVFCGDCRKKQDTPH

SEQ ID NO: 5 - protein, 221 aa, Homo sapiens
MEAGGFLDSLIYGACVVFTLGMFSAGLSDLRHMRMTRSVDNVQFLPFLTTEVNNLGWLSYGALKGDGILIVVNTVGAALQTLYILAYLHYCPRKRVVLLQTATLLGVLLLGYGYFWLLVPNPEARLQQLGLFCSVFTISMYLSPLADLAKVIQTKSTQCLSYPLTIATLLTSASWCLYGFRLRDPYIMVSNFPGIVTSFIRFWLFWKYPQEQDRNYWLLQT

SEQ ID NO: 6 - protein, 251 aa, Homo sapiens
MSDIGDWFRSIPAITRYWFAATVAVPLVGKLGLISPAYLFLWPEAFLYRFQIWRPITATFYFPVGPGTGFLYLVNLYFLYQYSTRLETGAFDGRPADYLFMLLFNWICIVITGLAMDMQLLMIPLIMSLLYVWAQLNRDMIVSFWFGTRFKACYLPWVILGFNYIIGGSVINELIGNLVGHLYFFLMFRYPMDLGGRNFLSTPQFLYRWLPSRRGGVSGFGVPPASMRRAADQNGGGGRHNWGQGFRLGDQ

SEQ ID NO: 7 - protein, 106 aa, Homo sapiens
MATPGPVIPEVPFEPSKPPVIEGLSPTVYRNPESFKEKFVRKTRENPVVPIGCLATAAALTYGLYSFHRGNSQRSQLMMRTRIAAQGFTVAAILLGLAVTAMKSRP

SEQ ID NO: 8 - protein, 78 aa, Homo sapiens
MGSGLPLVLLLTLLGSSHGTGPGMTLQLKLKESFLTNSSYESSFLELLEKLCLLLHLPSGTSVTLHHARSQHHVVCNT

SEQ ID NO: 9 - protein, 314 aa, Homo sapiens
MVAPVWYLVAAALLVGFILFLTRSRGRAASAGQEPLHNEELAGAGRVAQPGPLEPEEPRAGGRPRRRRDLGSRLQAQRRAQRVAWAEADENEEEAVILAQEEEGVEKPAETHLSGKIGAKKLRKLEEKQARKAQREAEEAEREERKRLESQREAEWKKEEERLRLEEEQKEEEERKAREEQAQREHEEYLKLKEAFVVEEEGVGETMTEEQSQSFLTEFINYIKQSKVVLLEDLASQVGLRTQDTINRIQDLLAEGTITGVIDDRGKFIYITPEELAAVANFIRQRGRVSIAELAQASNSLIAWGRESPAQAPA

SEQ ID NO: 10 - protein, 195 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 11 - protein, 462 aa, Homo sapiens [sequence omitted]

SEQ ID NO: 12 - protein, 247 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCKD

SEQ ID NO: 13 - protein, 113 aa, Homo sapiens
MNFYLLLASSILCALIVFWKYRRFQRNTGEMSSNSTALALVRPSSSGLINSNTDNNLAVYDLSRDILNNFPHSIARQKRILVNLSMVENKLVELEHTLLSKGFRGPSPHRKST

SEQ ID NO: 14 - protein, 365 aa, Homo sapiens
MGRWALDVAFLWKAVLTLGLVLLYYCFSIGITFYNKWLTKSFHFPLFMTMLHLAVIFLFSALSRALVQCSSHRARVVLSWADYLRRVAPTALATALDVGLSNWSFLYVTVSLYTMTKSSAVLFILIFSLIFKLEELRAALVLVVLLIAGGLFMFTYKSTQFNVEGFALVLGASFIGGIRWTLTQMLLQKAELGLQNPIDTMFHLQPLMFLGLFPLFAVFEGLHLSTSEKIFRFQDTGLLLRVLGSLFLGGILAFGLGFSEFLLVSRTSSLTLSIAGIFKEVCTLLLAAHLLGDQISLLNWLGFALCLSGISLHVALKALHSRGDGGPKALKGLGSSPDLELLLRSSQREEGDNEEEEYFVAQGQQ

SEQ ID NO: 15 - protein, 226 aa, Homo sapiens
MPTTKKTLMFLSSFFTSLGSFIVICSILGTQAWITSTIAVRDSASNGSIFITYGLFRGESSEELSHGLAEPKKKFAVLEILNNSSQKTLHSVTILFLVLSLITSLLSSGFTFYNSISNPYQTFLGPTGVYTWNGLGASFVFVTMILFVANTQSNQLSEELFQMLYPATTSKGTTHSYGYSFWLILLVILLNIVTVTIIIFYQKARYQRKQEQRKPMEYAPRDGILF

SEQ ID NO: 16 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 17 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 18 - protein, 193 aa, Homo sapiens [sequence omitted]

SEQ ID NO: 19 - DNA, 1146 bp, Homo sapiens
atgggtctgctccttcccctggcactctgcatcctagtcctgtgctgcggagcaatgtctccaccccagctggccctcaacccctcggctctgctctcccggggctgcaatgactccgatgtgctggcagttgcaggctttgccctgcgggatattaacaaagacagaaaggatggctatgtgctgagactcaaccgagtgaacgacgcccaggaatacagacggggtggcctgggatctctgttctatcttacactggatgtgctagagactgactgccatgtgctcagaaagaaggcatggcaagactgtggaatgaggatattttttgaatcagtttatggtcaatgcaaagcaatattttatatgaacaacccaagtagagttctctatttagctgcttataactgtactcttcgcccagtttcaaaaaaaaagatttacatgacgtgccctgactgcccaagctccatacccactgactcttccaatcaccaagtgctggaggctgccaccgagtctcttgcgaaatacaacaatgagaacacatccaagcagtattctctcttcaaagtcaccagggcttctagccagtgggtggtcggcccttcttactttgtggaatacttaattaaagaatcaccatgtactaaatcccaggccagcagctgttcacttcagtcctccgactctgtgcctgttggtctttgcaaaggttctctgactcgaacacactgggaaaagtttgtctctgtgacttgtgacttctttgaatcacaggctccagccactggaagtgaaaactctgctgttaaccagaaacctacaaaccttcccaaggtggaagaatcccagcagaaaaacacccccccaacagactccccctccaaagctgggccaagaggatctgtccaatatcttcctgacttggatgataaaaattcccaggaaaagggccctcaggaggcctttcctgtgcatctggacctaaccacgaatccccagggagaaaccctggatatttccttcctcttcctggagcctatggaggagaagctggttgtcctgcctttccccaaagaaaaagcacgcactgctgagtgcccagggccagcccagaatgccagccctcttgtccttccgcca

SEQ ID NO: 20 - DNA, 951 bp, Homo sapiens
atgtggctctacctggcggccttcgtgggcctgtactaccttctgcactggtaccgggagaggcaggtggtgagccacctccaagacaagtatgtctttatcacgggctgtgactcgggctttgggaacctgctggccagacagctggatgcacgaggcttgagagtgctggctgcgtgtctgacggagaagggggccgagcagctgaggggccagacgtctgacaggctggagacggtgaccctggatgttaccaagatggagagcatcgctgcagctactcagtgggtgaaggagcatgtgggggacagaggactctggggactggtgaacaatgcaggcattcttacaccaattaccttatgtgagtggctgaacactgaggactctatgaatatgctcaaagtgaacctcattggtgtgatccaggtgaccttgagcatgcttcctttggtgaggagagcacggggaagaattgtcaatgtctccagcattctgggaagagttgctttctttgtaggaggctactgtgtctccaagtatggagtggaagccttttcagatattctgaggcgtgagattcaacattttggggtgaaaatcagcatagttgaacctggctacttcagaacgggaatgacaaacatgacacagtccttagagcgaatgaagcaaagttggaaagaagcccccaagcatattaaggagacctatggacagcagtattttgatgccctttacaatatcatgaaggaagggctgttgaattgtagcacaaacctgaacctggtcactgactgcatggaacatgctctgacatcggtgcatccgcgaactcgatattcagctggctgggatgctaaatttttcttcatccctctatcttatttacctacatcactggcagactacattttgactagatcttggcccaaaccagcccaggcagtc

SEQ ID NO: 21 - DNA, 888 bp, Homo sapiens
atgagtgactccaaggaaccaagggtgcagcagctgggcctcctggggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaa

SEQ ID NO: 22 - DNA, 591 bp, Homo sapiens
atgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatggtactgtgtccggggattgcagccgttcgggcagggggcaagggctgctgtggtgctgggtgctgtggaaaccgctgcaggatgctgcgctcggtcttctcctcggcgttcggggtgcttggtgccatctactgcctctcggtgtctggagctgggctccgaaatggacccagatgcttaatgaacggcgagtggggctaccacttcgaagacaccgcgggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacccctcac

SEQ ID NO: 23 - DNA, 663 bp, Homo sapiens
atggaggcgggcggctttctggactcgctcatttacggagcatgcgtggtcttcacccttggcatgttctccgccggcctctcggacctcaggcacatgcgaatgacccggagtgtggacaacgtccagttcctgccctttctcaccacggaagtcaacaacctgggctggctgagttatggggctttgaagggagacgggatcctcatcgtcgtcaacacagtgggtgctgcgcttcagaccctgtatatcttggcatatctgcattactgccctcggaagcgtgttgtgctcctacagactgcaaccctgctaggggtccttctcctgggttatggctacttttggctcctggtacccaaccctgaggcccggcttcagcagttgggcctcttctgcagtgtcttcaccatcagcatgtacctctcaccactggctgacttggctaaggtgattcaaactaaatcaacccaatgtctctcctacccactcaccattgctacccttctcacctctgcctcctggtgcctctatgggtttcgactcagagatccctatatcatggtgtccaactttccaggaatcgtcaccagctttatccgcttctggcttttctggaagtacccccaggagcaagacaggaactactggctcctgcaaacc

SEQ ID NO: 24 - DNA, 753 bp, Homo sapiens
atgtcggacatcggagactggttcaggagcatcccggcgatcacgcgctattggttcgccgccaccgtcgccgtgcccttggtcggcaaactcggcctcatcagcccggcctacctcttcctctggcccgaagccttcctttatcgctttcagatttggaggccaatcactgccaccttttatttccctgtgggtccaggaactggatttctttatttggtcaatttatatttcttatatcagtattctacgcgacttgaaacaggagcttttgatgggaggccagcagactatttattcatgctcctctttaactggatttgcatcgtgattactggcttagcaatggatatgcagttgctgatgattcctctgatcatgtcagtactttatgtctgggcccagctgaacagagacatgattgtatcattttggtttggaacacgatttaaggcctgctatttaccctgggttatccttggattcaactatatcatcggaggctcggtaatcaatgagcttattggaaatctggttggacatctttattttttcctaatgttcagatacccaatggacttgggaggaagaaattttctatccacacctcagtttttgtaccgctggctgcccagtaggagaggaggagtatcaggatttggtgtgccccctgctagcatgaggcgagctgctgatcagaatggcggaggcgggagacacaactggggccagggctttcgacttggagaccag

SEQ ID NO: 25 - DNA, 318 bp, Homo sapiens
atggcgactcccggccctgtgattccggaggtcccctttgaaccatcgaagcctccagtcattgaggggctgagccccactgtttacaggaatccagagagtttcaaggaaaagttcgttcgcaagacccgcgagaacccggtggtacccataggttgcctggccacggcggccgccctcacctacggcctctactccttccaccggggcaacagccagcgctctcagctcatgatgcgcacccggatcgccgcccagggtttcacggtcgcagccatcttgctgggtctggctgtcactgctatgaagtctcgaccc

SEQ ID NO: 26 - DNA, 234 bp, Homo sapiens
atggggtctgggctgccccttgtcctcctcttgaccctccttggcagctcacatggaacagggccgggtatgactttgcaactgaagctgaaggagtcttttctgacaaattcctcctatgagtccagcttcctggaattgcttgaaaagctctgcctcctcctccatctcccttcagggaccagcgtcaccctccaccatgcaagatctcaacaccatgttgtctgcaacaca

SEQ ID NO: 27 - DNA, 942 bp, Homo sapiens
atggtggcgcctgtgtggtacttggtagcggcggctctgctagtcggctttatcctcttcctgactcgcagccggggccgggcggcatcagccggccaagagccactgcacaatgaggagctggcaggagcaggccgggtggcccagcctgggcccctggagcctgaggagccgagagctggaggcaggcctcggcgccggagggacctgggcagccgcctacaggcccagcgtcgagcccagcgggtggcctgggcagaagcagatgagaacgaggaggaagctgtcatcctagcccaggaggaggaaggtgtcgagaagccagcggaaactcacctgtcggggaaaattggagctaagaaactgcggaagctggaggagaaacaagcgcgaaaggcccagcgtgaggcagaggaggctgaacgtgaggagcggaaacgactcgagtcccagcgcgaagctgagtggaagaaggaggaggagcggcttcgcctggaggaggagcagaaggaggaggaggagaggaaggcccgcgaggagcaggcccagcgggagcatgaggagtacctgaaactgaaggaggcctttgtggtggaggaggaaggcgtaggagagaccatgactgaggaacagtcccagagcttcctgacagagttcatcaactacatcaagcagtccaaggttgtgctcttggaagacctggcttcccaggtgggcctacgcactcaggacaccataaatcgcatccaggacctgctggctgaggggactataacaggtgtgattgacgaccggggcaagttcatctacataaccccagaggaactggccgccgtggccaacttcatccgacagcggggccgggtgtccatcgccgagcttgcccaagccagcaactccctcatcgcctggggccgggagtcccctgcccaagccccagcc

SEQ ID NO: 28 - DNA, 585 bp, Homo sapiens
atggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgat

SEQ ID NO: 29 - DNA, 1386 bp, Homo sapiens
atgttggacttcgcgatcttcgccgttaccttcttgctggcgttggtgggagccgtgctctacctctatccggcttccagacaagctgcaggaattccagggattactccaactgaagaaaaagatggtaatcttccagatattgtgaatagtggaagtttgcatgagttcctggttaatttgcatgagagatatgggcctgtggtctccttctggtttggcaggcgcctcgtggttagtttgggcactgttgatgtactgaagcagcatatcaatcccaataagacattggacccttttgaaaccatgctgaagtcattattaaggtatcaatctggtggtggcagtgtgagtgaaaaccacatgaggaaaaaattgtatgaaaatggtgtgactgattctctgaagagtaactttgccctcctcctaaagctttcagaagaattattagataaatggctctcctacccagagacccagcacgtgcccctcagccagcatatgcttggttttgctatgaagtctgttacacagatggtaatgggtagtacatttgaagatgatcaggaagtcattcgcttccagaagaatcatggcacagtttggtctgagattggaaaaggctttctagatgggtcacttgataaaaacatgactcggaaaaaacaatatgaagatgccctcatgcaactggagtctgttttaaggaacatcataaaagaacgaaaaggaaggaacttcagtcaacatattttcattgactccttagtacaagggaaccttaatgaccaacagatcctagaagacagtatgatattttctctggccagttgcataataactgcaaaattgtgtacctgggcaatctgttttttaaccacctctgaagaagttcaaaaaaaattatatgaagagataaaccaagtttttggaaatggtcctgttactccagagaaaattgagcagctcagatattgtcagcatgtgctttgtgaaactgttcgaactgccaaactgactccagtttctgcccagcttcaagatattgaaggaaaaattgaccgatttattattcctagagagaccctcgtcctttatgcccttggtgtggtacttcaggatcctaatacttggccatctccacacaagtttgatccagatcggtttgatgatgaattagtaatgaaaactttttcctcacttggattctcaggcacacaggagtgtccagagttgaggtttgcatatatggtgaccacagtacttcttagtgtattggtgaagagactgcacctactttctgtggagggacaggttattgaaacaaagtatgaactggtaacatcatcaagggaagaagcttggatcactgtctcaaagagatat

SEQ ID NO: 30 - DNA, 741 bp, Homo sapiens
atgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaagggagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgtaaggac

SEQ ID NO: 31 - DNA, 339 bp, Homo sapiens
atgaacttctatttactcctagcgagcagcattctgtgtgccttgattgtcttctggaaatatcgccgctttcagagaaacactggcgaaatgtcatcaaattcaactgctcttgcactagtgagaccctcttcttctgggttaattaacagcaatacagacaacaatcttgcagtctacgacctctctcgggatattttaaataatttcccacactcaatagccaggcagaagcgaatattggtaaacctcagtatggtggaaaacaagctggttgaactggaacatactctacttagcaagggtttcagaggtgcatcacctcaccggaaatccacc

SEQ ID NO: 32 - DNA, 1095 bp, Homo sapiens
atggggaggtgggccctcgatgtggcctttttgtggaaggcggtgttgaccctggggctggtgcttctctactactgcttctccatcggcatcaccttctacaacaagtggctgacaaagagcttccatttccccctcttcatgacgatgctgcacctggccgtgatcttcctcttctccgccctgtccagggcgctggttcagtgctccagccacagggcccgtgtggtgctgagctgggccgactacctcagaagagtggctcccacagctctggcgacggcgcttgacgtgggcttgtccaactggagcttcctgtatgtcaccgtctcgctgtacacaatgaccaaatcctcagctgtcctcttcatcttgatcttctctctgatcttcaagctggaggagctgcgcgcggcactggtcctggtggtcctcctcatcgccgggggtctcttcatgttcacctacaagtccacacagttcaacgtggagggcttcgccttggtgctgggggcctcgttcatcggtggcattcgctggaccctcacccagatgctcctgcagaaggctgaactcggcctccagaatcccatcgacaccatgttccacctgcagccactcatgttcctggggctcttccctctctttgctgtatttgaaggtctccatttgtccacatctgagaaaatcttccgtttccaggacacagggctgctcctgcgggtacttgggagcctcttccttggcgggattctcgcctttggtttgggcttctctgagttcctcctggtctccagaacctccagcctcactctctccattgccggcatttttaaggaagtctgcactttgctgttggcagctcatctgctgggcgatcagatcagcctcctgaactggctgggcttcgccctctgcctctcgggaatatccctccacgttgccctcaaagccctgcattccagaggtgatggtggccccaaggccttgaaggggctgggctccagccccgacctggagctgctgctccggagcagccagcgggaggaaggtgacaatgaggaggaggagtactttgtggcccaggggcagcag

SEQ ID NO: 33 - DNA, 678 bp, Homo sapiens
atgcctaccacaaagaagacattgatgttcttatcaagctttttcaccagccttgggtccttcattgtaatttgctctattcttgggacacaagcatggatcaccagtacaattgctgttagagactctgcttcaaatgggagcattttcatcacttacggactttttcgtggggagagtagtgaagaattgagtcacggacttgcagaaccaaagaaaaagtttgcagttttagagatactgaataattcttcccaaaaaactctgcattcggtgactatcctgttcctggtcctgagtttgatcacgtcgctgctgagctctgggtttaccttctacaacagcatcagcaacccttaccagacattcctggggccgacgggggtgtacacctggaacgggctcggtgcatccttcgtttttgtgaccatgatactgtttgtggcgaacacgcagtccaaccaactctccgaagagttgttccaaatgctttacccggcaaccaccagtaaaggaacgacccacagttacggatactcgttctggctcatactgctcgtcattcttctaaatatagtcactgtaaccatcatcattttctaccagaaggccagataccagcggaagcaggagcagagaaagccaatggaatatgctccaagggacggaattttattc

SEQ ID NO: 34 - DNA, 387 bp, Homo sapiens
atggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccag

SEQ ID NO: 35 - DNA, 489 bp, Homo sapiens
atgcgacggctgctgatccctctggccctgtggctgggcgcggtgggcgtgggcgtcgccgagctcacggaagcccagcgccggggcctgcaggtggccctggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagc

SEQ ID NO: 36 - DNA, 579 bp, Homo sapiens
atgatccgctgcggcctggcctgcgagcgctgccgctggatcctgcccctgctcctactcagcgccatcgccttcgacatcatcgcgctggccggccgcggctggttgcagtctagcgaccacggccagacgtcctcgctgtggtggaaatgctcccaagagggcggcggcagcgggtcctacgaggagggctgtcagagcctcatggagtacgcgtggggtagagcagcggctgccatgctcttctgtggcttcatcatcctggtgatctgtttcatcctctccttcttcgccctctgtggaccccagatgcttgtcttcctgagagtgattggaggtctccttgccttggctgctgtgttccagatcatctccctggtaatttaccccgtgaagtacacccagaccttcacccttcatgccaaccgtgctgtcacttacatctataactgggcctacggctttgggtgggcagccacgattatcctgatcggctgtgccttcttcttctgctgcctccccaactacgaagatgaccttctgggcaatgccaagcccaggtacttctacacatctgcc

SEQ ID NO: 37 - DNA, 1502 bp, Homo sapiens
  CDS (37)...(1185)
acaaactgacccatcctgggccttgttctccacagaatgggtctgctccttcccctggcactctgcatcctagtcctgtgctgcggagcaatgtctccaccccagctggccctcaacccctcggctctgctctcccggggctgcaatgactccgatgtgctggcagttgcaggctttgccctgcgggatattaacaaagacagaaaggatggctatgtgctgagactcaaccgagtgaacgacgcccaggaatacagacggggtggcctgggatctctgttctatcttacactggatgtgctagagactgactgccatgtgctcagaaagaaggcatggcaagactgtggaatgaggatattttttgaatcagtttatggtcaatgcaaagcaatattttatatgaacaacccaagtagagttctctatttagctgcttataactgtactcttcgcccagtttcaaaaaaaaagatttacatgacgtgccctgactgcccaagctccatacccactgactcttccaatcaccaagtgctggaggctgccaccgagtctcttgcgaaatacaacaatgagaacacatccaagcagtattctctcttcaaagtcaccagggcttctagccagtgggtggtcggcccttcttactttgtggaatacttaattaaagaatcaccatgtactaaatcccaggccagcagctgttcacttcagtcctccgactctgtgcctgttggtctttgcaaaggttctctgactcgaacacactgggaaaagtttgtctctgtgacttgtgacttctttgaatcacaggctccagccactggaagtgaaaactctgctgttaaccagaaacctacaaaccttcccaaggtggaagaatcccagcagaaaaacacccccccaacagactccccctccaaagctgggccaagaggatctgtccaatatcttcctgacttggatgataaaaattcccaggaaaagggccctcaggaggcctttcctgtgcatctggacctaaccacgaatccccagggagaaaccctggatatttccttcctcttcctggagcctatggaggagaagctggttgtcctgcctttccccaaagaaaaagcacgcactgctgagtgcccagggccagcccagaatgccagccctcttgtccttccgccatgagaatcacacagagtcttctgtaggggtatggtgcgccgcatgacatgggaggcgatggggacgatggacagagacagagcgtgcacacgtagagtggctagtgaaggacgcctttttgactcttcttggtctcagcatgttgactgggattggaaataatgagactgagccctcggcttgggctgcactctaccctgtacactgccttgtaccctgagctgcatcacctcctaaactgagcagtctcataccatggagagatgcctctcttatgtcttcagccactcacttataaagatacttatcttttcagcagt

SEQ ID NO: 38 - DNA, 1349 bp, Homo sapiens
  CDS (111)...(1064)
agcagttggggcaggaggaagccgactgctgcctggtctgcaaagaagtcctttcaagtctctaggactggactcttcctaagcaagtccgagaaggaagcaccctcactatgtggctctacctggcggccttcgtgggcctgtactaccttctgcactggtaccgggagaggcaggtggtgagccacctccaagacaagtatgtctttatcacgggctgtgactcgggctttgggaacctgctggccagacagctggatgcacgaggcttgagagtgctggctgcgtgtctgacggagaagggggccgagcagctgaggggccagacgtctgacaggctggagacggtgaccctggatgttaccaagatggagagcatcgctgcagctactcagtgggtgaaggagcatgtgggggacagaggactctggggactggtgaacaatgcaggcattcttacaccaattaccttatgtgagtggctgaacactgaggactctatgaatatgctcaaagtgaacctcattggtgtgatccaggtgaccttgagcatgcttcctttggtgaggagagcacggggaagaattgtcaatgtctccagcattctgggaagagttgctttctttgtaggaggctactgtgtctccaagtatggagtggaagccttttcagatattctgaggcgtgagattcaacattttggggtgaaaatcagcatagttgaacctggctacttcagaacgggaatgacaaacatgacacagtccttagagcgaatgaagcaaagttggaaagaagcccccaagcatattaaggagacctatggacagcagtattttgatgccctttacaatatcatgaaggaagggctgttgaattgtagcacaaacctgaacctggtcactgactgcatggaacatgctctgacatcggtgcatccgcgaactcgatattcagctggctgggatgctaaatttttcttcatccctctatcttatttacctacatcactggcagactacattttgactagatcttggcccaaaccagcccaggcagtctaaagaaaactgggttggtgcttcttggaatgaaggcaaaaatctgaaattgttagtgtctcagtaatcctgatttagaacccaggctttttgtaacaatgtgttttcttgcctaaattcatttatctggcatcatcagagtactaacatgtttatatttcagatatccaaagcttaccactttaggtgatgaatctttactattttagcccttttttgatgagactatttgtctaaagtgaatcatttgttcttgccttattaaacagagtagatggaaaacaattt

SEQ ID NO: 39 - DNA, 1643 bp, Homo sapiens
  CDS (25)...(915)
aacatctggggacagcgggaaaacatgagtgactccaaggaaccaagggtgcagcagctgggcctcctggggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatagttgtttccctgctagcctcagcctccattgtggtatagcagaacttcacccacttgtaagccagcgcttcttctctccatccttggaccttcacaaatgccctgagacggttctctgttcgatttttcatcccctatgaacctgggtcttattctgtccttctgatgcctccaagtttccctggtgtagagcttgtgttcttggcccatccttggagctttataagtgacctgagtgggatgcatttagggggcgggcttggtatgttgtatgaatccactctctgttccttttggagattagactatttggattcatgtgtagctgccctgtcccctggggctttatctcatccatgcaaactaccatctgctcaacttccagctacaccccgtgcacccttttgactggggacttgctggttgaaggagctcatcttgcaggctggaagcaccagggaattaattcccccagtcaaccaatggcatccagagagggcatggaggctccatacaacctcttccacccccacatctttctttgtcctatacatgtcttccatttggctgtttctgagttgtagcctttataataaagtggtaaatgttgtaactgc

SEQ ID NO: 40 - DNA, 729 bp, Homo sapiens
  CDS (38)...(631)
actttcactcaccgcctgtccttcctgacacctcaccatgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatggtactgtgtccggggattgcagccgttcgggcagggggcaagggctgctgtggtgctgggtgctgtggaaaccgctgcaggatgctgcgctcggtcttctcctcggcgttcggggtgcttggtgccatctactgcctctcggtgtctggagctgggctccgaaatggacccagatgcttaatgaacggcgagtggggctaccacttcgaagacaccgcgggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacccctcactgaggctccactgaccgccgggttacacctgctccttcctggacgcctacctggctcgctcactcccttgctcgctagaataaactgctttgcgctctctt

SEQ ID NO: 41 - DNA, 1322 bp, Homo sapiens
  CDS (84)...(749)
gagccgcaggtctgggctgcagtaggtcccggcaaccgcaggctcgcggcgggcgctgggcgcgggatccgactctagtcgtaatggaggcgggcggctttctggactcgctcatttacggagcatgcgtggtcttcacccttggcatgttctccgccggcctctcggacctcaggcacatgcgaatgacccggagtgtggacaacgtccagttcctgccctttctcaccacggaagtcaacaacctgggctggctgagttatggggctttgaagggagacgggatcctcatcgtcgtcaacacagtgggtgctgcgcttcagaccctgtatatcttggcatatctgcattactgccctcggaagcgtgttgtgctcctacagactgcaaccctgctaggggtccttctcctgggttatggctacttttggctcctggtacccaaccctgaggcccggcttcagcagttgggcctcttctgcagtgtcttcaccatcagcatgtacctctcaccactggctgacttggctaaggtgattcaaactaaatcaacccaatgtctctcctacccactcaccattgctacccttctcacctctgcctcctggtgcctctatgggtttcgactcagagatccctatatcatggtgtccaactttccaggaatcgtcaccagctttatccgcttctggcttttctggaagtacccccaggagcaagacaggaactactggctcctgcaaacctgaggctgctcatctgaccactgggcaccttagtgccaacctgaaccaaagagacctccttgtttcagctgggcctgctgtccagcttcccaggtgcagtgggttgtgggaacaagagatgactttgaggataaaaggaccaaagaaaaagctttacttagatgattgattggggcctaggagatgaaatcactttttattttttagagatttttttttttaattttggaggttggggtgcaatctttagaatatgccttaaaaggccgggcgcggtggctcacgcctgtaatcccagcactttgggaggccaaggtgggcggatcgcctgaggtcaggagttcaagaccaacctgactaacatggtgaaaccccatctctactaaaaatacaaaattagccaggcatgatggcacatgcctgtaatcccagatacttgggaggctgaggcaggagaattgcttgaacccaggaggtggaggttgcagtgagctgagatcgtgccattgtgatatgaatatgccttatatgctgatatgaatatgccttaaaataaagtgttccccacccctgccc

SEQ ID NO: 42 - DNA, 3045 bp, Homo sapiens
  CDS (191)...(946)
gtttcgcctcagaaggctgcctcgctggtccgaattcggtggcgccacgtccgcccgtctccgccttctgcatcgcggcttcggcggcttccacctagacacctaacagtcgcggagccggccgcgtcgtgagggggtcggcacggggagtcgggcggtcttgtgcatcttggctacctgtgggtcgaagatgtcggacatcggagactggttcaggagcatcccggcgatcacgcgctattggttcgccgccaccgtcgccgtgcccttggtcggcaaactcggcctcatcagcccggcctacctcttcctctggcccgaagccttcctttatcgctttcagatttggaggccaatcactgccaccttttatttccctgtgggtccaggaactggatttctttatttggtcaatttatatttcttatatcagtattctacgcgacttgaaacaggagcttttgatgggaggccagcagactatttattcatgctcctctttaactggatttgcatcgtgattactggcttagcaatggatatgcagttgctgatgattcctctgatcatgtcactactttatgtctgggcccagctgaacagagacatgattgtatcattttggtttggaacacgatttaaggcctgctatttaccctgggttatccttggattcaactatatcatcggaggctcggtaatcaatgagcttattggaaatctggttggacatctttattttttcctaatgttcagatacccaatggacttgggaggaagaaattttctatccacacctcagtttttgtaccgctggctgcccagtaggagaggaggagtatcaggatttggtgtgccccctgctagcatgaggcgagctgctgatcagaatggcggaggcgggagacacaactggggccagggctttcgacttggagaccagtgaaggggcggcctcgggcagccgctcctctcaagccacatttcctcccagtgctgggtgcgcttaacaactgcgttctggctaacactgttggacctgacccacactgaatgtagtctttcagtacgagacaaagtttcttaaatcccgaagaaaaatataagtgttccacaagtttcacgattctcattcaagtccttactgctgtgaagaacaaataccaactgtgcaaattgcaaaactgactacattttttggtgtcttctcttctcccctttccgtctgaataatgggttttagcgggtcctagtctgctggcattgagctggggctgggtcaccaaacccttcccaaaaggacccttatctctttcttgcacacatgcctctctcccacttttcccaacccccacatttgcaactagaagaggttgcccataaaattgctctgcccttgacaggttctgttatttattgacttttgccaaggcttggtcacaacaatcatattcacgtaattttccccctttggtggcagaactgtagcaatagggggagaagacaagcagcggatgaagcgttttctcagcttttggaattgcttcgacctgacatccgttgtaaccgtttgccacttcttcagatatttttataaaaaagtaccactgagtcagtgagggccacagattggtattaatgagatacgagggttgttgctgggtgtttgtttcctgagctaagtgatcaagactgtagtggagttgcagctaacatgggttaggtttaaaccgtgggggatgcaacccctttgcgtttcatatgtaggcctactggctttgtgtagctggagtagttgggttgctttgtgttaggaggatccagatcatgttggctacagggagatgctctctttgagaggctcctgggcattgattccatttcaatctcattctggatatgtgttcattgagtaaaggaggagagaccctcatacgctatttaaatgtcacttttttgcctatcccccgttttttggtcatgtttcaattaattgtgaggaaggcgcagctcctctctgcacgtagatcattttttaaagctaatgtaagcacatctaagggaataacatgatttaaggttgaaatggctttagaatcatttgggtttgagggtgtgttattttgagtcatgaatgtacaagctctgtgaatcagaccagcttaaatacccacacctttttttcgtaggtgggcttttcctatcagagcttggctcataaccaaataaagttttttgaaggccatggcttttcacacagttattttattttatgacgttatctgaaagcagactgttaggagcagtattgagtggctgtcacactttgaggcaactaaaaaggcttcaaacgttttgatcagtttcttttcaggaaacattgtgctctaacagtatgactattctttcccccactcttaaacagtgtgatgtgtgttatcctaggaaatgagagttggcaaacaacttctcattttgaatagagtttgtgtgtacctctccatatttaatttatatgataaaataggtggggagagtctgaaccttaactgtcatgttttgttgttcatctgtggccacaataaagtttacttgtaaaattttagaggccattactccaattatgttgcacgtacactcattgtacaggcgtggagactcattgtatgtataagaatattctgacagtgagtgacccggagtctctggtgtaccctcttaccagtcagctgcctgcgagcagtcattttttcctaaaggtttacaagtatttagaactcttcagttcagggcaaaatgttcatgaagttattcctcttaaacatggttaggaagctgatgacgttattgattttgtctggattatgtttctggaataattttaccaaaacaagctatttgagttttgacttgacaaggcaaaacatgacagtggattctctttacaaattgaaaaaaataatccttattttgtataaaggacttccctttttgtaaactaatcctttttattggtaaaaattgtaaattaaaatgtgcaacttg

SEQ ID NO: 43 - DNA, 653 bp, Homo sapiens
  CDS (63)...(383)
atgaccttcaccgggaggctgaggtcggagtcccgattttctcctgctgctgtggcccggacatggcgactcccggccctgtgattccggaggtcccctttgaaccatcgaagcctccagtcattgaggggctgagccccactgtttacaggaatccagagagtttcaaggaaaagttcgttcgcaagacccgcgagaacccggtggtacccataggttgcctggccacggcggccgccctcacctacggcctctactccttccaccggggcaacagccagcgctctcagctcatgatgcgcacccggatcgccgcccagggtttcacggtcgcagccatcttgctgggtctggctgtcactgctatgaagtctcgaccctaagcccagggtctggccttgaaagctccgcagaaatgattccaaaacccagggagcaaccactggccctaccgtgggacttactccctcctctcctttgagaggcccatgtgtcgctggggaggaagtgaccctttgtgtaactgtaaccgaaagttttttcaaaaatcctagatgctgttgtttgaatgttacatacttctatttgtgccacatctcccctccactcccctgcttaataaactctaaaaatccacttgtatttaattcagt

SEQ ID NO: 44 - DNA, 439 bp, Homo sapiens
  CDS (75)...(311)
gtagaaacaggcctgttaaggagaggccaccgggacttcagtgtctcctccatcccaggagcgcagtggccactatggggtctgggctgccccttgtcctcctcttgaccctccttggcagctcacatggaacagggccgggtatgactttgcaactgaagctgaaggagtcttttctgacaaattcctcctatgagtccagcttcctggaattgcttgaaaagctctgcctcctcctccatctcccttcagggaccagcgtcaccctccaccatgcaagatctcaacaccatgttgtctgcaacacatgacagccattgaagcctgtgtccttcttggcccgggcttttgggccggggatgcaggaggcaggccccgaccctgtctttcagcaggcccccaccctcctgagtggcaataaataaaattcggtatgctg

SEQ ID NO: 45 - DNA, 1131 bp, Homo sapiens
  CDS (56)...(1000)
ctatgagatcccggcctcagggtggacgcagtggttctgcactgaggccctcgtcatggtggcgcctgtgtggtacttggtagcggcggctctgctagtcggctttatcctcttcctgactcgcagccggggccgggcggcatcagccggccaagagccactgcacaatgaggagctggcaggagcaggccgggtggcccagcctgggcccctggagcctgaggagccgagagctggaggcaggcctcggcgccggagggacctgggcagccgcctacaggcccagcgtcgagcccagcgggtggcctgggcagaagcagatgagaacgaggaggaagctgtcatcctagcccaggaggaggaaggtgtcgagaagccagcggaaactcacctgtcggggaaaattggagctaagaaactgcggaagctggaggagaaacaagcgcgaaaggcccagcgtgaggcagaggaggctgaacgtgaggagcggaaacgactcgagtcccagcgcgaagctgagtggaagaaggaggaggagcggcttcgcctggaggaggagcagaaggaggaggaggagaggaaggcccgcgaggagcaggcccagcgggagcatgaggagtacctgaaactgaaggaggcctttgtggtggaggaggaaggcgtaggagagaccatgactgaggaacagtcccagagcttcctgacagagttcatcaactacatcaagcagtccaaggttgtgctcttggaagacctggcttcccaggtgggcctacgcactcaggacaccataaatcgcatccaggacctgctggctgaggggactataacaggtgtgattgacgaccggggcaagttcatctacataaccccagaggaactggccgccgtggccaacttcatccgacagcggggccgggtgtccatcgccgagcttgcccaagccagcaactccctcatcgcctggggccgggagtcccctgcccaagccccagcctgaccccagtccttccctcttggactcagagttggtgtggcctacctggctatacatcttcatccctccccaccatcctggggaagtgatggtgtggccaggcagttatagattaaaggcctgtgagtactgct

SEQ ID NO: 46 - DNA, 1875 bp, Homo sapiens
  CDS (79)...(666)
ctcgctcgctcagagggaggagaaagtggcgagttccggatccctgcctagcgcggcccaacctttactccagagatcatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaaagcattcagtggaagtatatctatttttgtattttgcaaaatcatttgtaacagtccactctgtctttaaaacatagtgattacaatatttagaaagttttgagcacttgctataagttttttataacatcactagtgacactaataaaattaacttcttagaatgcatgatgtgtttgtgtgtcacaaatccagaaagtgaactgcagtgctgtaatacacatgttaatactgtttttcttctatctgtagttagtacaggatgaatttaaatgtgtttttcctgagagacaaggaagacttgggtatttcccaaaacaggtaaaaatcttaaatgtgcaccaagagcaaaggatcaacttttagtcatgatgttctgtaaagacaacaaatccctttttttttctcaattgacttaactgcatgatttctgttttatctacctctaaagcaaatctgcagtgttccaaagactttggtatggattaagcgctgtccagtaacaaaatgaaatctcaaaacagagctcagctgcaaaaaagcatattttctgtgtttctggactgcactgttgtccttgccctcacatagacactcagacaccctcacaaacacagtagtctatagttaggattaaaataggatctgaacattcaaaagaaagctttggaaaaaaagagctggctggcctaaaaacctaaatatatgatgaagattgtaggactgtcttcccaagccccatgttcatggtggggcaatggttatttggttattttactcaattggttactctcatttgaaatgagggagggacatacagaataggaacaggtgtttgctctcctaagagccttcatgcacacccctgaaccacgaggaaacagtacagtcgctagtcaagtggtttttaaagtaaagtatattcataaggtaacagttattctgttgttataaaactatacccactgcaaaagtagtagtcaagtgtctaggtctttgatattgctcttttggttaacactaagcttaagtagactatacagttgtatgaatttgtaaaagtatatgaacacctagtgagatttcaaacttgtaattgtggttaaatagtcattgtattttcttgtgaactgtgttttatgattttacctcaaatcagaaaacaaaatgatgtgctttggtcagttaataaaaatggttttacccact

SEQ ID NO: 47 - DNA, 1562 bp, Homo sapiens
  CDS (72)...(1460)
aaattgggccaggctgaggcgctgctgctggagcggccgatccgagacgtggctccctgggcggcagaaccatgttggacttcgcgatcttcgccgttaccttcttgctggcgttggtgggagccgtgctctacctctatccggcttccagacaagctgcaggaattccagggattactccaactgaagaaaaagatggtaatcttccagatattgtgaatagtggaagtttgcatgagttcctggttaatttgcatgagagatatgggcctgtggtctccttctggtttggcaggcgcctcgtggttagtttgggcactgttgatgtactgaagcagcatatcaatcccaataagacattggacccttttgaaaccatgctgaagtcattattaaggtatcaatctggtggtggcagtgtgagtgaaaaccacatgaggaaaaaattgtatgaaaatggtgtgactgattctctgaagagtaactttgccctcctcctaaagctttcagaagaattattagataaatggctctcctacccagagacccagcacgtgcccctcagccagcatatgcttggttttgctatgaagtctgttacacagatggtaatgggtagtacatttgaagatgatcaggaagtcattcgcttccagaagaatcatggcacagtttggtctgagattggaaaaggctttctagatgggtcacttgataaaaacatgactcggaaaaaacaatatgaagatgccctcatgcaactggagtctgttttaaggaacatcataaaagaacgaaaaggaaggaacttcagtcaacatattttcattgactccttagtacaagggaaccttaatgaccaacagatcctagaagacagtatgatattttctctggccagttgcataataactgcaaaattgtgtacctgggcaatctgttttttaaccacctctgaagaagttcaaaaaaaattatatgaagagataaaccaagtttttggaaatggtcctgttactccagagaaaattgagcagctcagatattgtcagcatgtgctttgtgaaactgttcgaactgccaaactgactccagtttctgcccagcttcaagatattgaaggaaaaattgaccgatttattattcctagagagaccctcgtcctttatgcccttggtgtggtacttcaggatcctaatacttggccatctccacacaagtttgatccagatcggtttgatgatgaattagtaatgaaaactttttcctcacttggattctcaggcacacaggagtgtccagagttgaggtttgcatatatggtgaccacagtacttcttagtgtattggtgaagagactgcacctactttctgtggagggacaggttattgaaacaaagtatgaactggtaacatcatcaagggaagaagcttggatcactgtctcaaagagatattaaaattttatacatttaaaatcattgttaaattgattgaggaaacaaccatttaaaaaaaatctatgttgaatccttttataaaccagtatcactttgtaatat

SEQ ID NO: 48 - DNA, 2030 bp, Homo sapiens
  CDS (171)...(914)
catttggggtttcggttcccccccttccccttccccggggtctgggggtgacattgcaccgcgcccctcgtggggtcgcgttgccaccccacgcggactccccagctggcgcgcccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgtaaggactgactacctggactgatcgcctgacagatcccacctgcctgtccactgcccatgactgagcccagccccagcccgggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtgcaggttgccaagaaaagggacctagtttagccattgccctggagatgaaattaatggaggctcaaggatagatgagctctgagtttctcagtactccctcaagactggacatcttggtctttttctcaggcctgagggggaaccatttttggtgtgataaataccctaaactgcctttttttcttttttgaggtggggggagggaggaggtatattggaactcttctaacctccttgggctatattttctctcctcgagttgctcctcatggctgggctcatttcggtccctttctccttggtcccagaccttgggggaaaggaaggaagtgcatgtttgggaactggcattactggaactaatggttttaacctccttaaccaccagcatccctcctctccccaaggtgaagtggagggtgctgtggtgagctggccactccagagctgcagtgccactggaggagtcagactaccatgacatcgtagggaaggaggggagatttttttgtagtttttaattggggtgtgggaggggcggggaggttttctataaactgtatcattttctgctgagggtggagtgtcccatccttttaatcaaggtgattgtgattttgactaataaaaaagaatttgt

SEQ ID NO: 49 - DNA, 493 bp, Homo sapiens
  CDS (98)...(439)
aaagtttcccaaatccaggcggctagaggcccactgcttcccaactaccagctgagggggtccgtcccgagaagggagaagaggccgaagaggaaacatgaacttctatttactcctagcgagcagcattctgtgtgccttgattgtcttctggaaatatcgccgctttcagagaaacactggcgaaatgtcatcaaattcaactgctcttgcactagtgagaccctcttcttctgggttaattaacagcaatacagacaacaatcttgcagtctacgacctctctcgggatattttaaataatttcccacactcaatagccaggcagaagcgaatattggtaaacctcagtatggtggaaaacaagctggttgaactggaacatactctacttagcaagggtttcagaggtccatcacctcaccggaaatccacctaaaagcgtacaggatgtaatgccagtggtggaaatcattaaagacactttgagtag

SEQ ID NO: 50 - DNA, 2044 bp, Homo sapiens
  CDS (288)...(1385)
agattccggcctggagctcccagggccgagcagaccttgggacctgtgagcgctgcatccaattaaccatgggaagggtcagcaccagccaccagccccttaggtgaggactctgcctggggctctgctgatggttccgaatcatggagctgcagagagctcctccagcctggagacgttcttggtgaaagctgtggtctaactccaccggctcttcctgcacattgtattcaagaggggtgcctgcccccgctgactcaggagctccggtgctgcagccgccacgaatggggaggtgggccctcgatgtggcctttttgtggaaggcggtgttgaccctggggctggtgcttctctactactgcttctccatcggcatcaccttctacaacaagtggctgacaaagagcttccatttccccctcttcatgacgatgctgcacctggccgtgatcttcctcttctccgccctgtccagggcgctggttcagtgctccagccacagggcccgtgtggtgctgagctgggccgactacctcagaagagtggctcccacagctctggcgacggcgcttgacgtgggcttgtccaactggagcttcctgtatgtcaccgtctcgctgtacacaatgaccaaatcctcagctgtcctcttcatcttgatcttctctctgatcttcaagctggaggagctgcgcgcggcactggtcctggtggtcctcctcatcgccgggggtctcttcatgttcacctacaagtccacacagttcaacgtggagggcttcgccttggtgctgggggcctcgttcatcggtggcattcgctggaccctcacccagatgctcctgcagaaggctgaactcggcctccagaatcccatcgacaccatgttccacctgcagccactcatgttcctggggctcttccctctctttgctgtatttgaaggtctccatttgtccacatctgagaaaatcttccgtttccaggacacagggctgctcctgcgggtacttgggagcctcttccttggcgggattctcgcctttggtttgggcttctctgagttcctcctggtctccagaacctccagcctcactctctccattgccggcatttttaaggaagtctgcactttgctgttggcagctcatctgctgggcgatcagatcagcctcctgaactggctgggcttcgccctctgcctctcgggaatatccctccacgttgccctcaaagccctgcattccagaggtgatggtggccccaaggccttgaaggggctgggctccagccccgacctggagctgctgctccggagcagccagcgggaggaaggtgacaatgaggaggaggagtactttgtggcccaggggcagcagtgaccagccagggcaaatggcttagaagcaggccactccccagcctgctgccagcactcactgtgctcaagccgccagggctcatcatggtagctgggagctgtggacgggagtcaccaggtggtggggccaagccagggactcatgacttttgcccctcccttcagagcctggtcacacaaggggcgagcaccaggccagcctgggactggccagagctgggcccaagctgcgctggaatcgcagcaggagaggggagtgggctggttcttcccaccacttcccaggctctgacagccgagactcatttccaaggcacagcagctttctaaagggactgagtttggactgggttttggacctccaggggctggagcttcatcacctgggcagtgtcttttctcagagagcaggtttctttatagtttggaaataaatggttcacggtccactggccgccttgtgttgctggagacgtgggggcagggaggggacagtgtgggcctggcctctcctttcctttccctgcctggagccttcttcaaatgtctggtcttaagccaggcctccttcattttctcgctcctgttagaacaccagtcccctccccagtggggccccactgcacctgctggcaggaaataaatgaatgtttactgagt

SEQ ID NO: 51 - DNA, 1043 bp, Homo sapiens
  CDS (157)...(837)
attagcataacccttcctcaggaagagtgagattttatatttgacaataaagtgttagactccatttctaaataccagacttcaaaagataaggttcaaaagtgttataagaagatattcctttttttgtcctagagaacttattttcctgtgaaaatgcctaccacaaagaagacattgatgttcttatcaagctttttcaccagccttgggtccttcattgtaatttgctctattcttgggacacaagcatggatcaccagtacaattgctgttagagactctgcttcaaatgggagcattttcatcacttacggactttttcgtggggagagtagtgaagaattgagtcacggacttgcagaaccaaagaaaaagtttgcagttttagagatactgaataattcttcccaaaaaactctgcattcggtgactatcctgttcctggtcctgagtttgatcacgtcgctgctgagctctgggtttaccttctacaacagcatcagcaacccttaccagacattcctggggccgacgggggtgtacacctggaacgggctcggtgcatccttcgtttttgtgaccatgatactgtttgtggcgaacacgcagtccaaccaactctccgaagagttgttccaaatgctttacccggcaaccaccagtaaaggaacgacccacagttacggatactcgttctggctcatactgctcgtcattcttctaaatatagtcactgtaaccatcatcattttctaccagaaggccagataccagcggaagcaggagcagagaaagccaatggaatatgctccaagggacggaattttattctgaattctctttcatctcattttggcgttgcatctattgtacatcagccctgagtagtaactggttagcttctctggacaattcagcatggtaacgtgactgtcatctgtgacagcatttgtgtttcatgacactgtgttcttcattgatgctgtactcctgaaaatttttcccacaaggttggggaaatgaatgggaaatgtcgctgg

SEQ ID NO: 52 - DNA, 971 bp, Homo sapiens
  CDS (29)...(418)
agacagcggcgggcgcaggacgtgcactatggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccaccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaacttt

SEQ ID NO: 53 - DNA, 695 bp, Homo sapiens
  CDS (73)...(564)
aagatttcagctgcgggacggtcaggggagacctccaggcgcagggaaggacggccagggtgacacggaagcatgcgacggctgctgatccctctggccctgtggctgggcgcggtgggcgtgggcgtcgccgagctcacggaagcccagcgccggggcctgcaggtggccctggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagctaagccagcactgagctgcgtggtgcctccaggaccgctgccggtggtaaccagtggaagaccccagcccccagggagaggaccccgttctatccccagccatgataataaagctgctctcccagctgcctctc

SEQ ID NO: 54 - DNA, 1914 bp, Homo sapiens
  CDS (80)...(661)
actctctgctgtcgcccgtcccgcgcgctcctccgacccgctccgctccgctccgctcggccccgcgccgcccgtcaacatgatccgctgcggcctggcctgcgagcgctgccgctggatcctgcccctgctcctactcagcgccatcgccttcgacatcatcgcgctggccggccgcggctggttgcagtctagcgaccacggccagacgtcctcgctgtggtggaaatgctcccaagagggcggcggcagcgggtcctacgaggagggctgtcagagcctcatggagtacgcgtggggtagagcagcggctgccatgctcttctgtggcttcatcatcctggtgatctgtttcatcctctccttcttcgccctctgtggaccccagatgcttgtcttcctgagagtgattggaggtctccttgccttggctgctgtgttccagatcatctccctggtaatttaccccgtgaagtacacccagaccttcacccttcatgccaaccgtgctgtcacttacatctataactgggcctacggctttgggtgggcagccacgattatcctgatcggctgtgccttcttcttctgctgcctccccaactacgaagatgaccttctgggcaatgccaagcccaggtacttctacacatctgcctaacttgggaatgaatgtgggagaaaatcgctgctgctgagatggactccagaagaagaaactgtttctccaggcgactttgaacccattttttggcagtgttcatattattaaactagtcaaataagctaaaataatttgggagaaaatattttttaagtagtgttatagtttcatgtttatcttttattatgttttgtgaagttgtgtcttttcactaattacctatactatgccaatatttccttatatctatccataacatttatactacatttgtaagagaatatgcacgtgaaacttaacactttataaggtaaaaatgaggtttccaagatttaataatctgatcaagttcttgttatttccaaatagaatggacttggtctgttaagggctaaggagaagaggaagataaggttaaaagttgttaatgaccaaacattctaaaagaaatgcaaaaaaaaagtttattttcaagccttcgaactatttaaggaaagcaaaatcatttcctaaatgcatatcatttgtgagaatttctcattaatatcctgaatcattcatttcagctaaggcttcatgttgactcgatatgtcatctaggaaagtactatttcatggtccaaacctgttgccatagttggtaaggctttcctttaagtgtgaaatatttagatgaaattttctcttttaaagttctttatagggttagggtgtgggaaaatgctatattaataaatctgtagtgttttgtgtttatatgttcagaaccagagtagactggattgaaagatggactgggtctaatttatcatgactgatagatctggttaagttgtgtagtaaagcattaggagggtcattcttgtcacaaaagtgccactaaaacagcctcaggagaataaatgacttgcttttctaaatctcaggtttatctgggctctatcatatagacaggcttctgatagtttgcaactgtaagcagaaacctacatatagttaaaatcctggtctttcttggtaaacagattttaaatgtctgatataaaacatgccacaggagaattcggggatttgagtttctctgaatagcatatatatgatgcatcggataggtcattatgattttttaccatttcgacttacataatgaaaaccaattcattttaaatatcagattattattttgtaagttgtggaaaaagctaattgtagttttcattatgaagttttcccaataaaccaggtattct

SEQ ID NO: 55 - DNA, 20 bp, Homo sapiens
gatcccgggtcacgtgggat

SEQ ID NO: 56 - DNA, 16 bp, Homo sapiens
atcccacgtgacccgg

SEQ ID NO: 57 - protein, 367 aa, Homo sapiens
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCTVFQTQPVTSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 58 - protein, 317 aa, Rattus rattus
MWLYLLALVGLWNLLRLFRERKVVSHLQDKYVFITGCDSGFGNLLARQLDRRGMRVLAACLTEKGAEQLRSKTSDRLETVILDVTKTESIVAATQWVKERVGNRGLWGLVNNAGISVPVGPNEWMRKKDFASVLDVNLLGVIEVTLNMLPLVRKARGRVVNIASTMGRMSLVGGGYCISKYGVEAFSDSLRRELTYFGVKVAIIEPGGFKTNVTNMERLSDNLKKLWDQTTEEVKEIYGEKFQDSYMKAMESLVNTCSGDLSLVTDCMEHALTSCHPRTRYSPGWDAKFFYLPMSYLPTFLSDAVIHWGSVKPARAL

SEQ ID NO: 59 - protein, 356 aa, Homo Sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDC

SEQ ID NO: 60 - protein, 23 aa, Homo sapiens
  VARIANT (1)...(23): Xaa = Any Amino Acid
IYQXLTXLKAAVGELXXXSKXQX

SEQ ID NO: 61 - protein, 202 aa, Homo sapiens
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 62 - protein, 221 aa, Mus musculus
MEAGGVADSFLSSACVLFTLGMFSTGLSDLRHMQRTRSVDNIQFLPFLTTDVNNLSWLSYGVLKGDGTLIIVNSVGAVLQTLYILAYLHYSPQKHGVLLQTATLLAVLLLGYGYFWLLVPDLEARLQQLGLFCSVFTISMYLSPLADLAKIVQTKSTQRLSFSLTIATLFCSASWSIYGFRLRDPYIAVPNLPGILTSLIRLGLFCKYPPEQDRKYRLLQT

SEQ ID NO: 63 - protein, 245 aa, Cephalobidae elegans
MDLENFLLGIPIVTRYWFLASTIIPLLGRFGFINVQWMFLQWDLVVNKFQFWRPLTALIYYPVTPQTGFHWLMMCYFLYNYSKALESETYRGRSADYLFMLIFNWFFCSGLCMALDIYFLLEPMVISVLYVWCQVNKDTIVSFWFGMRFPARYLPWVLWGFNAVLRGGGTNELVGILVGHAYFFVALKYPDEYGVDLISTPEFLHRLIPDEDGGIHGQDGNIRGARQQPRGHQWPGGVGARLGGN

SEQ ID NO: 64 - protein, 241 aa, Cephalobidae elegans
MRRNARRRVNRDEQEDGFVNHMMNDGEDVEDLDGGAEQFEYDEDGKKIGKRKAAKLQAKEEKRQMREYEVREREERKRREEEREKKRDEERAKEEADEKAEEERLRKEREEKERKEHEEYLAMKASFAIEEEGTDAIEGEEAENLIRDFVDYVKTNKVVNIDELSSHFGLKSEDAVNRLQHFIEEGLVQGVMDDRGKFIYISDEEFAAVAKFINQRGRVSIHEIAEQSNRLIRLETPSAAE

SEQ ID NO: 65 - protein, 239 aa, Sus scrofa
MAAEDVVATGADPSDLESGGLLHEIFTSPLNLLLLGLCIFLLYKIVRGDQPAASGDSDDDMAAEDVAATGADPSELEGGGLLHEIFTSPLNLLLLGLCIFLLYKIVRGDQPAASDSDDDEPPPLPRLKRRDFTPAELRRFDGVQDPRILMAINGKVFDVTKGRKFYGPEGPYGVFAGRDASRGLATFCLDKEALKDEYDDLSDLTPAQQETLNDWDSQFTFKYHHVGKLLKEGEEPTVY

SEQ ID NO: 66 - protein, 499 aa, Macaca fascicularis
MLDFAIFAVTFLLALVGAVLYLYPASRQAAGIPGITPTEEKDGNLPDIVNSGSLHEFMDLIPDLAVETWLLLAVTLVLLYLYGTHSHGLFKKLGIPGPTPLPLLGNILSYRKGFWTDMECYKKYGKVWGFYDGRQPVLAITDPNMIKTVLVKECYSVFTNRRPFGPVGFMKNAISIAEDEEWKRIRSLLSPTFTSGKLKEMVPIIAKYGDVLVRNLRREAETGKPVTLKDVFGAYSMDVITSTSFGVNIDSLNNPQDPFVENTKKLLRFDFLDPFFLSITIFPFIIPILEVLNISIFPREVTSFLRKSVKRIKESRLKDTQKHRVDFLQLMIDSQNSKETESHKALSDLELVAQSIIFIFAGYETTSSVLSFIIYELATHPDVQQKLQEEIDTVLPNKAPPTYDTVLQMEYLDMVSKKNNDNIDPYIYTPFGSGPRNCIGMRFALMNMKLAIIRVLQNFSFKPCKETQIPLKLRLGGLLQTEKPIVLKIESRDGTVSGA

SEQ ID NO: 67 - protein, 457 aa, Saccharomyces cerevisiae
MGRWALDVAFLWKAVLTLGLVLLYYCFSIGITFYNKWLTKSFHFPLFMTMLHLAMNRTVFLAFVFGWYFCSIALSIYNRWMFDPKDGLGIGYPVLVTTFHQATLWLLSGIYIKLRHKPVKNVLRKNNGFNWSFFLKFLLPTAVASAGDIGLSNVSFQYVPLTIYTIIKSSSIAFVLLFGCIFKLEKFHWKLALSVIIMFVGVALMVFKPSDSTSTKNDQALVIFGSFLVLASSCLSGLRWVYTQLMLRNNPIQTNTAAAVEESDGALFTENEDNVDNEPVVNLANNKMLENFGESKPHPIHTIHQLAPIMGITLLLTSLLVEKPFPGIFSSSIFRLDTSNGGVGTETTVLSIVRGIVLLILPGFAVFLLTICEFSILEQTPVLTVSIVGIVKELLTVIFGIIILSERLSGFYNWLGMLIIMADVCYYNYFRYKQDLLQKYHSVSTQDNRNELKGFQD